(19) United States
(12) Patent Application Publication
(10) Pub. No.: US 2022/0096318 A1
(43) Pub. Date: Mar. 31, 2022

(54) DEVICE, SYSTEM AND METHOD FOR REDUCING HEADACHE PAIN

(71) Applicant: THE REGENTS OF THE UNIVERSITY OF CALIFORNIA, Oakland, CA (US)

(72) Inventors: Ronald M. Harper, Los Angeles, CA (US); Eberhardt K. Sauerland, Reno, NV (US)

(21) Appl. No.: 17/497,796

(22) Filed: Oct. 8, 2021

Related U.S. Application Data
(63) Continuation-in-part of application No. 16/100,362, filed on Aug. 10, 2018.
Application No. 16/100,362 is a continuation of application No. 14/546,784, filed on Nov. 18, 2014, now Pat. No. 10,045,907.
Application No. 14/546,784 is a continuation-in-part of application No. PCT/US20/27227, filed on Apr. 8, 2020.
(60) Provisional application No. 61/905,616, filed on Nov. 18, 2013.
(60) Provisional application No. 62/831,152, filed on Apr. 8, 2019.

(51) Int. Cl. A61H 23/02; A61H 21/00; A61M 21/00

(52) U.S. Cl. CPC A61H 23/02; A61H 2205/027; A61M 21/00; A61H 2201/0184; A61H 2201/5002; A61H 2201/5005; A61H 2201/501; A61H 2201/5012; A61H 2201/5097; A61M 2021/0022; A61M 2205/18; A61M 2205/3553; A61M 2205/3584; A61M 2205/3592; A61M 2210/0662; A61H 2201/0107; A61H 21/00

(57) ABSTRACT

A device for stimulating at least one cranial nerve and/or spinal nerve is described. The device includes a vibratory motor, and an earpiece, wherein the earpiece is molded substantially to fit within the external ear canal and contacting the concha of a subject's ear. The present invention provides a method of reducing migraine headache, trigeminal neuropathy pain, normalize breathing, normalize blood pressure, induce sleep, increase salivation, improve vertigo, nausea, and visual dysfunction. A method for non invasive neuromodulation includes the steps of positioning a vibratory earpiece within an external ear canal of a subject, applying vibrational energy through the vibratory earpiece to stimulate mechanoreceptors of sensory fibers on cranial nerves 5, 7, 9 and 10, and cervical nerves C2 and C3, and regulating the subject's breathing and blood pressure simultaneously based on the stimulation. Further, the present invention provides a method for treating sleep-disturbed breathing and autonomic disorders.

### CROSS-REFERENCE TO RELATED APPLICATIONS

This application is a continuation-in-part of U.S. patent application Ser. No. 16/100,362, filed Aug. 10, 2018, which claims priority to U.S. patent application Ser. No. 14/546,784, filed Nov. 18, 2014, which claims priority to U.S. Provisional Patent Application No. 61/905,616 filed Nov. 18, 2013, the subject matter of which are each incorporated herein by reference in their entireties. Further, this application is a continuation-in-part of International Patent Application No. PCT/US20/27227, filed Apr. 8, 2020, which claims priority to U.S. Provisional Patent Application No. 62/831,152, filed Apr. 8, 2019, the subject matter of which are each incorporated herein by reference in their entireties.

### BACKGROUND OF THE INVENTION

Migraine headaches affect approximately 9% (21 million female; 7 million male) of the population in the United States. The worldwide estimate of incidence is 650 million. There are a number of different forms of migraine. A principal characteristic of all migraines is recurrence, and the pain can be moderate to severe if left untreated. For example, the headache may be unilateral, can be triggered by light, sound, smells, or other stimuli, and is often accompanied by nausea, vestibular symptoms, and visual distortions, or preceded by an aura.

Interventions for migraine are difficult, since the pain may involve multiple central nervous system structures and neurotransmitters. In many instances, the autonomic nervous system is involved, which leads to nausea, vomiting, and cardiovascular signs, as well as headache. Other substantial variations in migraine appear, ranging from ocular pain accompanied by vision loss or blindness, muscle tension, sinus pain, and vertebrobasilar pain associated with vertigo. A variation of migraine pain is generally called “trigeminal neuropathy,” and typically includes long-lasting oral pain, often arising from failed or compromised dental procedures, with trigeminal nerve (cranial nerve 5), facial nerve (cranial nerve 7), glossopharyngeal nerve (cranial nerve 9), or vagal (cranial nerve 10) irritation involved. The disorder can be accompanied by severe pain for many years, paraesthesias (such as tickling, odd sensations, or numbness), or sensations of burning. The so-called “burning mouth” syndrome, characterized by long-lasting, severe pain in the tongue or oral cavity i.e., stomatodynia and glossodynia, as well as severe, overwhelming pain from mild stimulation to the face, trigeminal neuralgia, also appears. These variations emphasize the complexity of migraine and associated pain syndromes.

Classical approaches to pain intervention include pharmacologic intervention, electrical stimulation, including transcranial electrical stimulation (DaSilva et al., 2012, The Journal of Head and Face Pain, 52: 1283-1295), electrical stimulation of the forehead, deep brain structures, or nerves within the brain or deep to the surface (Meng et al., 2013, Neurology, 81: 1102-1103; Mosqueira et al., 2013, Rev Neurol, 57(2): 57-63; Schoenen et al., 2013, Neurology, 0(8): 697-704), and Botox application (Silberstein et al., 2000; The Journal of Head and Face Pain, 40: 445-450) to reduce innervation to scalp muscles. Electrical stimulation of the peripheral skin poses a risk of injury to the underlying tissue, and can be uncomfortable. Outcomes of muscle paralysis procedures from Botox are often transient, and require skilled professional intervention, with careful attention to infection risk. The stimulation procedures requiring deep brain stimulation or access to nerves involve major surgery with substantial anesthetic and infection concerns (Pedersen et al., 2013; Cephalagia, 33(14): 1179-1193). Other procedures can vary in effectiveness. A range of pharmaceutical interventions exist (Olesen and Ashina, 2011, Trends in Pharmacological Sciences, 33(6): 352-359), and include serotonin agonists and nitric oxide antagonists. Many of these pharmaceutical interventions have severe side effects, including excessive sleepiness and addiction, especially to opioid agents. The impaired cognitive, arousal, gastric irritation, vestibular, and affective consequences of pharmaceutical agents pose concerns. Trigeminal neuralgia typically requires seizure medication, vascular decompression surgery, or lesioning of areas of the 5th cranial nerve (Janetta, 1980, Ann Surg, 192(4): 518-525). The invasiveness and severity of side effects make none of these options optimal.

Since migraine headaches involve multiple central nervous system (CNS) modulators, these classical approaches to pain intervention are not sufficiently targeted. There is thus a need for a migraine pain intervention via stimulation of multiple cranial nerves in a pattern that interferes with pain perception. Ideally, these stimuli would be non-invasive, and be patient-controllable, with the patient adjusting the stimuli when pain appears. The patient should also have the option to “condition” CNS processes to suppress migraine development, i.e., apply stimulation to “train” the brain to suppress brain activity that might lead to later migraine onset.

Further, there exists a significant need to facilitate breathing and support blood pressure in a range of medical disorders. In addition, a variety of medical conditions require control over autonomic nervous system regulation. The respiratory-related syndromes are accompanied by such characteristics as diminished activation of all muscles of respiration (hypoventilation), obstructed air passage by reduced activation of upper airway muscles, especially during sleep (obstructive sleep apnea, OSA) (Harper, R M et al., 1978, Chapter 14 in “Sleep Apnea Syndromes”, C. Guilleminault and W. Dement, Eds., Alan R. Liss, Inc., New York), or insufficient activation of thoracic, abdominal, or other respiratory musculature, such as in spinal cord injury or in generalized clonic-tonic epilepsy seizures. Additionally, patients with compromised action of smooth muscle air passages, such as those found with chronic obstructive pulmonary disease (COPD), or an impaired autonomic nervous system, including Phox2B mutations resulting in congenital central hypoventilation syndrome (CCHS), require intervention. CCHS subjects lose drive to all respiratory muscles during sleep. Another autonomic nervous system concern is Multiple System Atrophy; patients typically show obstructed breathing or hypoventilation during sleep or at rest. A group of patients with inherited disorders that involve progressive muscle weakness and loss of muscle tissue, termed muscular dystrophy (MD), also are frequently afflicted with disordered breathing during sleep. A major concern in heart failure patients is the presence of central apnea, a cessation of action of both diaphragmatic and upper airway musculature. This respiratory disorder is usually expressed as periodic or Cheyne-Stokes breathing in heart failure; that is, periods of breathing followed by cessation of all respiratory efforts for epochs of a minute or more; both Cheyne-Stokes and obstructive sleep apnea occur in over half of heart failure patients.

Another autonomic condition of concern is postural orthostatic tachycardia syndrome (POTS), a condition typically characterized by low resting blood pressure and impaired or deficient rise in blood pressure occurs with blood-pressure elevating behaviors. A device in such cases that normalizes resting blood pressure, and modifies the gain of the baroreflex to prevent postural collapse would benefit the art. Yet another autonomic regulation condition amenable to intervention by the proposed device is Cyclic Vomiting Syndrome (CVS), a condition of episodes of repeated vomiting and nausea that can last for hours or days (Bhandari, S. Jha et al., 2018, diagnosis, and treatment Clinical Autonomic Research, 28:203-209). CVS is sometimes called “intestinal migraine.” The syndrome is sometimes treated with drugs for migraine, and is exacerbated with strong affect, particularly positive emotions, a clue that suggests a central brain origin, and susceptible to intervention by a device that has been used for classic migraine pain.

### Incidence of Conditions:

The number of patients requiring supplementary ventilatory assistance in the United States alone is very large; approximately 20% of males and 10% of females are affected by obstructive sleep apnea, with the numbers rising with increased obesity. There are substantial numbers of pediatric cases, as well. Typical OSA numbers for the US approximate 34 million cases. The number of patients with partial or complete spinal cord injury in the US is also substantial, with approximately 12,000 new cases annually. Of these cases, a large number show sleep-disordered breathing, with conservative estimates of pediatric spinal cord injury patients (30%) showing obstructive apnea or hypoventilation at night; an additional sizeable number require assisted ventilation even during daylight hours. Although the number of cases of congenital central hypoventilation syndrome, a condition resulting from mutations in the PHOX2B gene, is small, (approximately 300 US cases), the costs for support for affected patients who typically require continuous nocturnal ventilation, and in some cases, 24 hour ventilatory assistance, is substantial. The failure to provide adequate nocturnal ventilation results in an exceptionally short survival rate, with a very large number of deaths occurring in teenage years. Between 25,000 and 100,000 Americans have Multiple Systems Atrophy, which is accompanied by hypoventilation, stridor, and obstructive sleep apnea, with sudden death a frequent outcome. Myotonic dystrophy has a prevalence estimated by genetic testing to be as high as 1/1000 in some populations; several recent studies suggest that sleep-disordered breathing can contribute to disease characteristics in these populations. Heart failure is the most common cause of admission in hospitals for patients over the age of 65 in the US, and affects approximately 5.7 million people in the U.S.; half of these heart failure patients show sleep-disordered breathing, and disrupted cardiovascular control accompanying the disrupted respiratory control. The prevalence of Cyclic Vomiting Syndrome is unclear, with estimates from 0.04 to 1.9% children (Bhandari, S. Jha et al., 2018, diagnosis, and treatment Clinical Autonomic Research, 28:203-209); however, recent evidence suggests that the prevalence in adults has been underestimated. The collective data suggest that the need for inexpensive, non-invasive autonomic and ventilatory support for all of these patient groups is very great, amounting to tens of millions of patients in the US alone.

### Sources of Drive to Breathing—Traditional:

Respiration is principally driven by the need to supply oxygen (O₂) to tissue and to remove carbon dioxide (CO₂) as a metabolic by-product of tissue metabolism. Levels of O₂ and CO₂ are normally precisely maintained by sensors located in the brain and in discrete sites in the principal arteries of the neck and aorta. Increased CO₂ leads to a perception of “air hunger,” and results in pronounced increases in ventilation; low Oz also leads to increased ventilation, but that drive is somewhat more complex.

### Sources of Drive to Breathing—Non-Traditional:

There is increasing evidence that other, non-chemical, i.e., not CO₂ or O₂, drives to breathe can assist respiration. Children with congenital central hypoventilation syndrome (CCHS) lack central chemosensation, and do not respond to increased CO₂ levels. However, if those children exercise in conditions that would normally increase CO₂ and deplete O₂, e.g., playing soccer, they ventilate normally (Gozal, D. et al., 1996, Am. J. Respir. Crit. Care Med., 153:761-768; Paton, J. Y. et al., 1993, Am. Rev. Respir. Dis., 147:1185-1191). These movements induced by exercise increase breathing. The relationship between limb movement and enhanced ventilation has been repeatedly demonstrated (Eldridge, F. L. et al., 1985, Respir. Physiol., 59:313-337), and may result from afferent activity caused by peripheral limb action to activate the central brain areas associated with breathing. This outcome has been demonstrated by functional magnetic resonance imaging studies of peripheral foot movement (Macey, P. M. et al., 2007, Society for Neuroscience Abstracts, 230.3). The mechanisms underlying the coupling of peripheral limb action with breathing are unclear, but likely derive from the need to provide immediate oxygenation for locomotor muscle action for escape, rather than waiting for accumulation of CO₂ to stimulate breathing. It is apparent from these data that ancillary sources of breathing stimulation exist, and these sources can be used to enhance ventilation. This concept has been used to develop a proprioceptive stimulation device that has successfully assisted breathing in premature infants (Kesavan, K. et al., 2016, PLoS ONE, 11(6):e0157349), and spinal cord injury patients (Woo, M S et al., 2014, Am J Respir Crit Care Med, 189:A1280). (and see Ronald M. Harper et al. PCT International Application No. PCT/US14/47642, filed Jul. 22, 2014, for Device, System and Method for Facilitating Breathing via Simulation of Limb Movement. Published Jan. 29, 2015).

In the same fashion, circumstances such as sleep can distort normal regulation of blood pressure, leading to loss of perfusion; those blood pressure changes often accompany breathing pauses, but can occur independently of breathing. The blood pressure regulatory systems also depend on sensory input, especially from fields located in the auditory meatus, the sensory fields of cranial nerves 5, 7, 9, and 10; cranial nerve 9 plays an especially important role, with its direct projections to the baroreceptor system.

Other non-chemical processes that interfere with breathing timing or extent:

Although reduced drive from chemo-sensation leads to impaired drive to breathe, many other mechanisms can lead to breathing failure. Obstructive sleep apnea patients show only modest CO₂ chemo-sensitive loss, as do heart failure, spinal cord or COPD patients. Heart failure patients, however, have deficits in timing of afferent signals from carotid chemoreceptors, a consequence of alterations in circulation time resulting from their primary circulatory condition, leading to the periodic or Cheyne-Stokes breathing.

Obstructive sleep apnea patients principally show a “disconnect” between activation of upper airway muscles and the diaphragm, i.e., diaphragmatic movements continue, but upper airway muscle actions are lost (Harper, R M et al., 1978, Chapter 14 in “Sleep Apnea Syndromes”, C. Guilleminault and W. Dement, Eds., Alan R. Liss, Inc., New York). Depending on segmental injury level, spinal cord patients show a reduced overall activation of the respiratory musculature, including the upper airway (Woo, M S et al., 2014, Am J Respir Crit Care Med, 189; A1280). A common process underlying many of these disturbed breathing patterns is a failure of timing or extent of drive to all airway muscles, or in the case of obstructive sleep apnea, a cluster of airway muscles. The device described here can overcome those deficits by providing exaggerated drive to specialized sensory systems that affect breathing timing and extent of drive to the different groups of respiratory muscles, and do that by stimulating branches of the associated nerves located in the auditory meatus; these nerves share branches in the oro-pharyngeal cavity.

Sensory Nerves Influencing Breathing Patterns:

The different sensory nerves influencing breathing patterns are complex, but can be summarized into several categories.

Chemo-sensing: The principal sensory nerves for sensing CO₂ and O₂ levels include nerves from sensors in the aorta and carotid bodies which principally carry information on O₂ levels in the blood and on the pressure exerted by the blood on the inner walls of the arteries (blood pressure). The latter is an extremely important factor in respiratory timing; elevation of blood pressure suppresses breathing (Trelease, R B et al., 1985, Experimental Neurology 90:173-186), principally to the upper airway muscles, but also to the diaphragm, and lowering of blood pressure enhances inspiratory efforts and breathing rate. Sensing of low O₂ provides overall facilitation of breathing. The carotid bodies also have a minor role in CO₂ sensing. A large part of CO₂ sensing occurs in central brain regions, and will not be considered further here. Critical to the discussion below, the sensory signals for blood pressure, O₂ and CO₂ signals are passed to the brain respiratory regulatory areas by a branch of the glossopharyngeal nerve (cranial nerve 9) for the carotid bodies, and cranial nerve 10 (vagus) for the aorta (see FIGS. 1A and 1B) (Agur, A M R Dailey et al., 2006, Grant's Atlas of Anatomy, Eleventh Edition, Williams and Wilkens, 2006). Cranial nerve 9 receives afferents from the carotid body, as well as sensory fibers from the posterior oral cavity and upper pharynx. The fibers in the oral cavity pass airflow information centrally, and the sinus nerve conducts baroreceptor and chemoreceptor fibers centrally as well. However, nerve 9 sends a tympanic branch (A), supplying the medial (inner) portion of the tympanic membrane which will be the focus of vibratory stimulation discussed here. Cranial nerve 10 (vagus) carries baroreceptor sensor information from the heart (FIG. 1B) (C) and both motor and sensory fibers of the pharynx and sensory fibers of the soft palate (FIG. 1B) (B). All of these sensory processes are important for breathing, including airflow and proprioceptive fibers from pharyngeal muscles. Those sensory processes, in sensing airflow, trigger central brain processes to elicit inspiratory and expiratory efforts. (As described later in various embodiments of the device and method described herein, the significance for vibratory stimulation of the auditory canal is that cranial nerves 5 (3^{rd} division), 9, and 10 provide sensory nerve innervation for that canal (FIG. 1B) (A), and in that role can be excited to “trick” the brain into signaling that airflow occurs). The cervical nerves C2 and C3 also are stimulated by vibration of the auditory canal; C3 contributes to the phrenic nerve, the principal nerve driving the diaphragm, the major muscle for respiration, and cervical nerve C2 provides essential sensory information of movement of accessory respiratory muscles.

Lung stretch receptors: Significant breathing timing signals arrive to brain breathing drive areas from stretch receptors, sensing lung inflation. On inspiration, these signals project to the respiratory phase-switching area of the parabrachial pons of the brainstem, signaling the brain to shut off inspiration when approaching appropriate lung inflation; cranial nerve 10, the vagus nerve, carries those signals (FIG. 1B) (D), and stimulation within the auricular vagus in the ear canal can trigger those inspiratory-expiratory phase switching signals. Forceful breathing efforts will also trigger accessory respiratory muscles of the upper thorax and cervical regions; cervical fibers of C2 and C3 also serve the external ear, and will interpret vibration of their sensory fields as a need to continue breathing efforts.

Oral, nasal, and pharyngeal airflow sensors. The movement of air through nasal, oral, and pharyngeal passages stimulates airflow receptors, and sensory activity from those receptors plays a vital role in facilitating breathing efforts. The sensory nerves for air movement are in trigeminal (cranial nerve 5, FIG. 1C,) afferents, as well as in cranial nerves 7, 9, and 10 (FIG. 1E, FIG. 1A, FIG. 1B). FIG. 1C illustrates the potential for interaction with auditory canal vibratory stimulation and those afferents; the anterior auricular nerve (A), a branch of the third division of 5, serves the external ear and ear canal, while afferents in the second division of 5 (D) and third divisions of 5 (C), carry sensory information of airflow from the oral cavity (E), and also provide motor innervation (C) for the oral musculature. In addition, the chorda tympani (B) carries sensory information from the tongue, and travels with the third division of the trigeminal nerve. More detail of the chorda tympani is shown in FIG. 1E, which also illustrates the close interactions between facial nerve branches and the geniculate ganglion of 7 in a pathological situation, with eruption of herpes zoster in the external auditory canal (Appendix 1, FIG. 1E (C)). The pathology extends to the posterior portion of the tragus (the small, pointed eminence of the external ear, situated in front of the concha; FIG. 1E (D)). The close interaction of nerves of cranial nerve 7 in the oral cavity conveyed by the chorda tympani are shown in FIG. 1E (B). The statements on innervation and anatomy drawings are derived from Grant's Atlas of Anatomy (Agur, AMR. Dailey et al., 2006, Grant's Atlas of Anatomy, Eleventh Edition, Williams and Wilkens).

Proprioceptive fibers of the tongue and other oral airway muscles: The genioglossal fibers of the tongue, as well as a number of oral airway muscles, including the tensor and levator palati, masseter, medial pterygoid, temporalis, and superior constrictor muscles, discharge phasically with the respiratory cycle (Anch, A M et al., 1981, Clin. Neurophysiol., 21:317-3301981; Hairston, L E et al., 1978, Anat. Rec., 190:411; Hairston, L E et al., 1981, Clin. Neurophysiol., 21:287-297; Hairston, L E et al., 1981, Clin. Neurophysiol., 21:299-306; Harper, R M et al., 1978, Chapter 14 in “Sleep Apnea Syndromes”, C. Guilleminault and W. Dement, Eds., Alan R. Liss, Inc., New York; Remmers, J E et al., 1978, Chapter 13 in “Sleep Apnea Syndromes,” C. Guilleminault and W. Dement, Eds. Alan Liss, Inc., New York; Remmers, J E et al., 1978, J. Appl. Physiol. 44:931-938); these muscles lead to closure of the mandible and thus assist in airway dilation. The most well-studied of these muscles is the genioglossus, (FIG. 1D), the principal oral dilator for breathing (Harper, R M et al., 1978, Chapter 14 in “Sleep Apnea Syndromes”, C. Guilleminault and W. Dement, Eds., Alan R. Liss, Inc., New York). Movement of all of those muscles is accompanied by proprioceptive sensory discharge, which is carried by their respective sensory fibers to respiratory regulatory sites, in addition to brain areas controlling other oral airway activities, including mastication and swallowing. The proprioceptive action is essential for timing and extent of upper airway muscle action. The cranial nerves carrying proprioceptive fibers include cranial nerve 12 (Neuhuber, W et al., 1980, Anat. Embryol., 158:349-360), which is also the principal motor nerve for the genioglossal fibers of the tongue (FIG. 1D, (B)). Those sensory fibers are presumably conveyed to the nodose ganglion (FIG. 1D, (A)), although some of those fibers may pass through the second division of the trigeminal nerve to the mesencephalic nucleus of V. The proprioceptive fibers are carried, in addition to 5, in cranial nerves 7, 9, and 10 (FIG. 1A through FIG. 1C and FIG. 1E), of these, cranial nerves 5, 10, and 12 most likely carry proprioceptive fibers, but both 7 and 9 also contain sensory fibers, some of which convey proprioceptive input.

Sensory Nerve Summary: Numerous sensory nerves participate in timing of the respiratory cycle and in modifying the extent of ventilatory efforts. Those nerves mediate multiple actions in addition to chemosensation, and are especially important for timing of onset of inspiratory effort relative to diaphragmatic efforts. The sensory modes involve blood pressure, lung inflation, air movement, and proprioception, in addition to classical CO₂ and O₂ sensing. Adequate functioning to maintain appropriate ventilation requires exquisitely precise interactions from multiple systems, in which timing is of essential importance.

Accordingly, there is a need in the art for alternative, non-invasive, devices and procedures that are easy to use for reducing headache pain and alleviating symptoms associated with migraine pain and devices and methods to facilitate breathing and support blood pressure while also regulating autonomic outflow that rely on the interactions described above to restore adequate function. The present invention satisfies this need.

### SUMMARY OF THE INVENTION

In one aspect, the present invention is related to a method for non-invasive neuromodulation comprising: positioning a vibratory earpiece configured to contact an external ear of a subject; applying vibrational energy through the vibratory earpiece to stimulate mechanoreceptors of sensory fibers on cranial nerves 5, 7, 9 and 10 and cervical nerves C2 and C3; and regulating the subject's breathing and blood pressure simultaneously based on the stimulation. In one embodiment, the method further comprises regulating autonomic outflow based on the stimulation of cranial nerves which also contain parasympathetic autonomic fibers and influence sympathetic outflow. In one embodiment, mechanoreceptors are stimulated simultaneously by the vibrational energy. In one embodiment, the application of vibrational energy is applied through at least a portion of the skin of at least one of the auditory canal, auricle, and concha of the subject's ear. In one embodiment, the stimulating is indicative of nerve sensations for airflow, upper airway muscle positioning, chemoreception, and blood pressure changes. In one embodiment, the stimulating elicits reflexive motor actions to activate upper airway muscles, diaphragmatic muscles, ancillary thoracic musculature, and abdominal breathing musculature. In one embodiment, the stimulating reduces blood pressure of the subject for regulating blood pressure. In one embodiment, the stimulating increases blood pressure of the subject for regulating blood pressure. In one embodiment, the stimulating enhances breathing extent of the subject, and reduces breathing variability. In one embodiment, the stimulating regulates autonomic outflow of the subject. In one embodiment, the method is used for treating autonomic disorders. In one embodiment, the method is used for treating sleep-disturbed breathing of a periodic pattern, obstructed upper airway, central, or hypoventilatory nature. In one embodiment, the method is used for treating headache disorders. In one embodiment, the method is used for treating vestibular disorders.

In one aspect, the present invention relates to a device for non-invasive neuromodulation comprising: an earpiece comprising a housing configured to contact a subject's external ear; a vibratory element connected to the housing, wherein the vibratory element transmits vibrational energy to an outer wall of housing; and a controller configured to generate a stimulation signal for stimulating one or more mechanoreceptors of sensory fibers of cranial nerves 5, 7, 9 and 10 and cervical nerves C2 and C3 when the housing is positioned in or around the external ear canal for simultaneously regulating the subject's breathing and blood pressure. In one embodiment, the stimulation signal is configured to regulate autonomic outflow. In one embodiment, the stimulation signal is configured to stimulate mechanoreceptors simultaneously. In one embodiment, the stimulation signal is configured to reduce blood pressure of the subject for regulating blood pressure. In one embodiment, the stimulation signal is configured to increase blood pressure of the subject for regulating blood pressure. In one embodiment, the stimulation signal is configured to enhance breathing of the subject. In one embodiment, the vibratory element is embedded within the housing. In one embodiment, the vibratory element is releasably connected to the housing.

In one aspect the present invention relates to a method for treating autonomic disorders or sleep-disturbed breathing comprising: positioning the above device into or around at least one external ear canal of a subject; and applying the vibrational energy.

In one aspect, the present invention relates to a method for treating sleep-disturbed breathing and autonomic disorders comprising: applying vibrational stimulus to sensory nerves for simultaneous regulation of blood pressure, respiration, headache, and vestibular disorders.

In one aspect, the present invention relates to a system for treating sleep-disturbed breathing and autonomic disorders comprising: a vibratory element operationally coupled to a controller, wherein the controller is configured to send a stimulus signal to the vibratory element capable of stimulating sensory nerves for simultaneous regulation of blood pressure and respiration.

In one aspect, the present invention relates to a method of reducing headache pain in a subject, comprising: contacting the ear of a subject with device comprising a vibratory element; transmitting vibrational energy via the device to the subject's ear; and stimulating at least one nerve in the subject's ear via the vibrational energy, thereby reducing headache pain in the subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is a diagram of cranial nerve 9. FIG. 1B is a diagram of cranial nerve 10. FIG. 1C is a diagram of cranial nerve 5. FIG. 1D is a diagram of cranial nerve 12. FIG. 1E is a diagram of cranial nerve 7.

FIG. 2 is a schematic illustration of an exemplary programmable control unit and earpiece, coupled by an insulated wire.

FIG. 3 is a photograph of an exemplary programmable control unit with two earpieces, coupled by insulated wire, and demonstrating the positons of patient-controllable up and down buttons in the middle surface of the control that can control the intensity of vibration of the vibrating motors. The control unit is programmed using a computing device to frequency, overall intensity, patterning of vibrating pulses, and overall duration of stimulation session.

FIG. 4A and FIG. 4B, are schematic illustrations of the frontal view (FIG. 4A) and top view (FIG. 4B) of an exemplary vibrating device with the internal vibrating motor and rod of the present invention. The rod is used to transfer vibration to the deepest recesses of the auditory canal.

FIG. 5 is a schematic illustration of an exemplary vibrating device with metal rod for increased vibratory intensity inserted into the external ear canal. This version will be used in patients who require vibration that extends optimally to the deepest recesses of the auditory canal.

FIG. 6 is a schematic illustration of an exemplary vibrating device of the present invention with the vibrating motor and an air duct, but without an internal vibrating rod. In certain embodiments where the rod is absent, the vibrations are carried evenly to all parts of the device. The air duct ensures an identical atmospheric pressure on the outside and the inside of the inserted device.

FIG. 7A and FIG. 7B, are schematic illustrations of an exemplary vibratory earpiece with an air duct.

FIG. 8 is a schematic illustration of an exemplary vibrating device, comprising an air duct, inserted into the external ear canal. The air duct allows for the establishment of air pressure equilibrium.

FIG. 9 is a schematic illustration of an exemplary device, where the earpiece comprises a disc magnet at its proximal end which is able to be magnetically coupled to a magnet of opposite polarity attached to a vibratory motor. Thus, in this embodiment, the motor need not be embedded or housed within the earpiece itself, but rather can be releasably coupled to the earpiece as desired.

FIG. 10 is a schematic illustration of an exemplary device, where the earpiece comprises a disk magnet at its proximal end which is magnetically coupled to a magnet of opposite polarity attached to a vibratory motor. This figure shows that the two disc magnets have precisely latched on to each other, and established a firm connection for vibratory energy transfer.

FIG. 11A and FIG. 11B, depicts a subject in the process of attaching the two device parts without visual control. FIG. 11A shows the inserted earpiece with the embedded disc magnet and opening for the air canal. The motor with its magnet of opposite polarity is close but not yet attached. FIG. 11B shows that the magnetic vibrator has precisely latched onto its target.

FIG. 12 is a schematic of a cross-sectional (coronal) view through the external ear canal and auricle, showing the major structures affected by the device of the present invention.

FIG. 13A and FIG. 13B, is an illustration of sensory input from the ear to the brain through multiple cranial (cranial nerves, CN 5, 7, 9, and 10), and C2 and C3 spinal nerves. The figure also shows that the sensory input for pain projects to a common nucleus, the spinal nucleus of 5 and spinal tract. The common input assists in confusing the brain into misperceiving a pain stimulus by using the proposed vibration to stimulate the multiple nerves simultaneously with an innocuous vibration. The ear is shown in coronal view.

FIG. 14A and FIG. 14B, is another illustration of sensory input from the ear to the brain, and illustrates the large number of cranial nerves (5, 7, 9, and 10), and spinal nerves (C2 and C3) that can be stimulated with the device, with a number of those cranial nerves represented on the concha as well as the ear canal. The external ear and the ear canal are shown in lateral view.

FIG. 15A is a flow chart of a method for non-invasive neuromodulation according to one embodiment. FIG. 15B is a diagram of a device for non-invasive neuromodulation according to one embodiment.

FIG. 16A shows a diagram of the carotid sinus nerve, a 9th nerve branch that extends from the carotid baroreceptors (blood pressure sensors); branches of the 9th nerve also serve sensory fields near the tympanic membrane, and thus are affected by mechanical vibration delivered by the device in the auditory canal (the brain will be unable to differentiate stimulation of branches of the 9th nerve fibers from the carotid sinus and tympanic nerve, thus allowing modulation of blood pressure. FIG. 16B shows a diagram of a silicon impression 302 in one auditory meatus with embedded and bent metal probe in black to carry vibrations from a vibratory motor 304, which is powered through electrical wires from a remote 3 volt power supply; the metal probe having a bend since the auditory canal often follows a curved path sometimes nearly at right angles, from the external surface to the tympanic membrane, and the bar must follow that path; and to the right a silicon impression and vibratory motor in situ of a patient is shown according to one embodiment.

FIG. 17 is a graph depicting the results of the most substantial reported pain reduction for 18 different subjects with migraine or trigeminal neuropathy pain after use of the device for less than 50 minutes of the present invention. Of the 18 subjects, 17 showed reduction in reported pain, and one showed no change. In some cases, the pain reduction was substantial (e.g., on a pain scale of 0 to 10, where a rating of 10 is so severe that the subject is at risk to go unconscious, 9 to 0 on two subjects, 10 to 2 on one subject, 8 to 0, 8 to 1, and 8 to 2 on three additional subjects). That outcome is statistically significant (p<0.0001, paired t-test).

FIG. 18 is a graph depicting the reduction of pain reported by a subject with severe migraine after use of the device of the present invention on 6 successive occasions; the dates of interventions are shown below the graph.

FIG. 19 is a graph depicting the reduction of pain reported by a subject with severe migraine after use of the device of the present invention on 12 successive occasions.

FIG. 20 is a graph depicting the reduction of pain reported by a subject with trigeminal neuropathy after use of the device of the present invention on 4 successive occasions.

FIG. 21 is a graph depicting the reduction of pain reported by a subject with burning mouth syndrome after use of the device of the present invention on 9 successive occasions. Further interventions have ended since pain has been resolved.

FIG. 22 is a set of traces, depicting the decline of beat-by-beat systolic blood pressure in a severely hypertensive patient with concurrent migraine during stimulation. Vertical red lines represent consecutive minutes (minute intervals vary, since baseline and stimulation periods differ, and an attempt was made to maximize collection time). “Low Stim” refers to mild vibration at 1.5 volts; “High Stim” refers to stronger vibration at 3 volts. BP declines to a nadir 4 minutes after low stim onset, remains low with high stimulation, and rises again after 14 minutes post stim. Migraine pain declined during treatment as well. All measures were taken with the patient in a sitting, unmoving position.

FIG. 23 is a set of traces depicting the effect of stimulation on the beat-by-beat mean arterial blood pressure of a subject with orthostatic hypotension (characterized by syncope with head turning or arising from a seated position). The condition was resolved with the intervention to the point that the subject was able to abandon wheelchair use. The top “Baseline” recording depicts the low, but wildly varying blood pressure at rest. The middle “Low Amp Stim” tracing recording depicts beat-by-beat blood pressure at low stimulation (1.5 volts to vibratory motor); blood pressure is mildly elevated from baseline, but significantly stabilizes by the tenth minute of stimulation. The lowest tracing depicts beat-by-beat blood pressure at high stimulation (3.0 volts to vibratory motor). Blood pressure continues to be elevated over baseline conditions, with no wild fluctuations.

FIG. 24 is a set of recordings depicting the effect of stimulation on the cardiac R-R intervals (time between successive heart beats; an index of both rate and variability), of a subject with orthostatic hypotension. The top recording depicts R-R intervals at baseline and shows a relatively slow rate (long intervals), and excessive slow variation, typical of intermittent sympathetic nervous system action. The middle tracing depicts R-R intervals at low stimulation (1.5 volts to vibratory motor); the tracing shows an overall faster rate (shorter intervals), and substantial periods of highly rhythmical respiratory-related variation, especially between minutes 5-7. Such respiratory modulation is mediated by the vagus (cranial nerve 10) and is considered protective against cardiac arrhythmia. The third recording depicts R-R intervals post-stimulation, with the cardiac patterning returning to highly variable large slow variation typically found with sympathetic action, and indicative of risk.

FIG. 25A is a graph showing a decline in mean respiratory rate in breaths/minute with auditory canal vibratory stimulation in 31 subjects over a 30-minute period according to one embodiment. FIG. 25B is a graph showing respiratory variability declined following a no-stim baseline and following stimulation (High Stim) with variability recovered post stimulation according to one embodiment.

FIG. 26A is a graph showing respiratory traces of disturbed breathing in a congenital central hypoventilation (CCHS) patient (No Vibration) according to one embodiment which is corrected by vibratory stimulation (Vibration). FIG. 26B is a graph showing periodic breathing in obstructive sleep apnea (No Vibration) according to one embodiment, and correction of that breathing pattern by the vibratory device (Vibration).

FIG. 27A through FIG. 27C show Systolic (FIG. 27A), Diastolic (FIG. 27B) and Mean Arterial Pressure (MAP) (FIG. 27C) during no vibration baseline, following low- and high-level vibration of the auditory canal, and after a second Post Stimulation) baseline with no vibration (total session time was 30 minutes) according to one embodiment.

FIG. 28 is a graph of blood pressure values showing how vibratory stimulation normalizes blood pressure in those who have mildly low blood pressure according to one embodiment.

FIG. 29 is a graph showing beat-by-beat systolic (SYS), mean (MAP), and diastolic (Dias) blood pressure during vibration and during CPAP (onset at arrow) according to one embodiment; CPAP was unable to maintain blood pressure in this hypertensive CCHS patient (CCHS patients show very high sympathetic tone; thus, they have overall high blood pressures). (MAP=Mean Arterial Pressure).

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical devices, systems and methods for reducing headache and trigeminal neuropathy (oral-facial) pain and to facilitate breathing and support sleep pressure in a range of medical disorders. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Description

The present invention includes a device, system and method that disrupt or inhibit central nervous system processes that mediate pain in the head or face by activating both the cranial nerve mediating that pain as well as other cranial nerves that use the same central nuclei which integrate pain processing. Since migraine headaches involve multiple central nervous system (CNS) modulators, the possibility exists to interfere with pain processes by distorting sensory input so as to confuse neural mechanisms normally mediating pain. The potential for confusing such processes is readily demonstrated by the poor brain discrimination of intense pain, such as that from a myocardial infarction (“heart attack”). The pain from the heart in such an attack is often referred to the arm, ear, jaw, or shoulder; such poor discrimination offers the possibility that other sensory input could confuse the brain for perception of pain. In one embodiment, the method of the present invention can abolish obstructive and periodic breathing in all subjects, creating a slow, deep, minimally-variant breathing pattern. In one embodiment, the method of present invention may be used to treat sleep-disturbed breathing.

The present invention utilizes mechanical vibration (as opposed to electrical stimulation) to activate these nerves through skin sensory receptors and mask pain perception. The vibratory stimuli of the present invention are non-invasive and patient-controllable, such that the subject may make real-time adjustments to the amplitude and timing of the stimuli when pain appears. In some embodiments, the subject may “teach” or “condition” selected CNS processes to suppress migraine development. For example, the subject may apply vibration to “train” the brain to suppress brain activity that would otherwise lead to later migraine onset. Further, as described elsewhere herein, vibrational activation of these cranial nerves also serves to treat hyposalivation, hypotension, hypertension, and visual and vestibular disturbances related to migraine in subjects in need.

Much of cranial pain, including oral pain, is mediated by the 5^{th} cranial (trigeminal) nerve which integrates pain through one of its nuclei, the descending spinal nucleus. That nucleus also mediates pain from other cranial nerves, 7, 9, and 10, as well as from two spinal (cervical) nerves of the posterior scalp, C2 and C3. It should be appreciated that each or all of these cranial and cervical nerves can contribute to the sensation of pain in various forms of migraine. As contemplated herein, the device and system of the present invention activates sensory fibers of cranial nerves 5, 7, 9, and 10, and cervical nerves C2, and C3, and disrupts activity of cranial nerve 5 by masking pain in its descending nucleus, or in insular cortical and other brain sites that integrate pain and other sensory signals, including vibration signals. Activity of the other cranial nerves can be similarly disrupted. Such stimulation of 7, 9, 10, C2, and C3 has traditionally not been considered feasible, because these nerves are largely inaccessible, lying deep below the skin surface and being widely dispersed over the head and neck. However, the present invention is at least partially based on the discovery that a site exists where those nerves are in close proximity and innervate the surface in one area. As demonstrated herein, that area lies within the external auditory canal and extends to the auricle and concha. In fact, this site for administration of vibratory stimuli is the only site in the body in which the cranial nerves 5, 7, 9, 10 and spinal nerves C2 and C3 converge at an easily-accessible skin surface.

Vibrations produced by the device, system, and method of the present invention activate cutaneous mechanoreceptors in the external ear and ear canal. These tactile corpuscles (Meissner corpuscles) respond rapidly to mechanical skin changes, such as vibrations. The resulting nerve activities (in the form of electrical action potentials) enter the brain stem. Subsequently, they appear to inhibit incoming pain stimuli from various regions of the head and neck.

Accordingly, the present invention includes a method of stimulating one or more sensory fibers of at least one of cranial nerves 5, 7, 9, and 10, and spinal nerves C2, and C3, comprising applying vibrational energy to at least a portion of the skin of at least one of the auditory canal, auricle and concha of a subject's ear. In another embodiment, the present invention includes a method of reducing headache or trigeminal neuropathy pain in a subject, comprising applying vibrational energy to at least a portion of the skin of at least one of the auditory canal, auricle and concha of the subject's ear, thereby stimulating one or more sensory fibers of at least one of cranial nerves 5, 7, 9, and 10, and spinal nerves C2, and C3. In another embodiment, multiple cranial nerves and/or spinal nerves are stimulated simultaneously. In one embodiment, headache or trigeminal neuropathy pain is reduced within 1 to 100 minutes after application of vibratory stimuli. For example, in certain embodiments, headache pain is reduced within 50 minutes, within 30 minutes, within 10 minutes, within 9 minutes, 8 minutes, within 7 minutes, within 6 minutes, within 5 minutes, within 4 minutes, within 3 minutes, within 2 minutes, or even within 1 minute. In certain embodiments, the headache pain is reduced in less than 1 minute.

The present invention makes use of the neuroanatomical principle that cranial nerves 7, 9, 10, and spinal nerves C2 and C3 use the spinal descending nucleus of cranial nerve 5 for pain mediation, allowing activation of nerves from 7, 9, 10, C2 and C3 to mask pain signals from cranial nerve 5. Accordingly, the multiplicity of cranial and spinal nerves allows the devices and methods of the present invention to mask pain signals from cranial nerve 5. In another embodiment, if pain results not from cranial nerve 5, but from C2 or C3 areas, as it may from tension of the neck muscles or the dura of the brain, or from regions supplied by 7, 9, or 10, as it may from sites in the face, oral cavity or pharynx, activation of sensory fibers in the remaining cranial or spinal nerves can also provide a similar masking effect. Cranial nerve 9, for example, mediates glossopharyngeal neuralgia (excruciating pain similar to trigeminal neuralgia, but with origins in the posterior oral cavity), and is also a target for the present invention, since vibration would excite nearby fibers in 5, 7, 10 and C2-C3 which could mask such glossopharyngeal neuralgia. Accordingly, the present invention provides a novel approach for masking glossopharyngeal neuralgia pain, and has been used in that context In one embodiment, the device and system of the present invention includes an earpiece, within which is contained a small vibrating motor (diameter 8-10 mm). In one embodiment, the vibrating motor is attached to a small metal rod (5-15 mm length, 2-4 mm diameter), angled in the caudal direction to accommodate the anatomy of the auditory canal. In one embodiment, the earpiece comprises an air duct running the length of the earpiece to allow for the establishment of air pressure equilibrium. In one embodiment, the motor is not contained within the earpiece itself, but is instead releasably coupled to the earpiece, for example using magnetic or adhesive coupling.

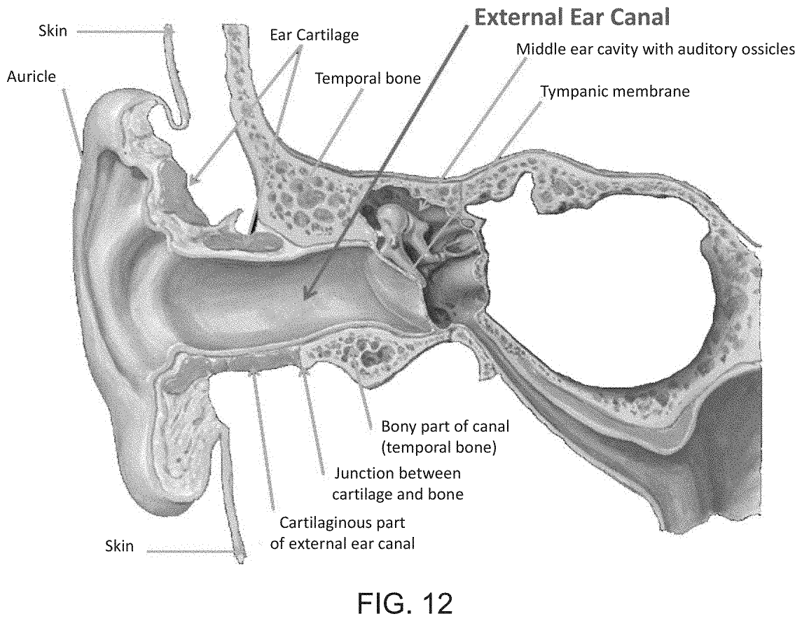

It should be appreciated that there are no limitations to the actual shape and/or dimensions of the earpiece molding, vibrating motor, rod, and duct. Moreover, in one embodiment, to reduce the extent of vibration to the innermost portions of the auditory canal, the rod can be eliminated. Preferably, the device fits within the subject's ear such that the tip of the device extends immediately beyond the junction of the cartilaginous part of the ear canal and the temporal bone (the “Junction” in FIG. 12), but short of the tympanic membrane. While not required, custom molding is preferred, because the configurations of ear canals may differ greatly from subject to subject, and appropriate contact with the skin tissue inside the canal and concha is needed to enhance activation of the sensory nerves innervating the tissue.

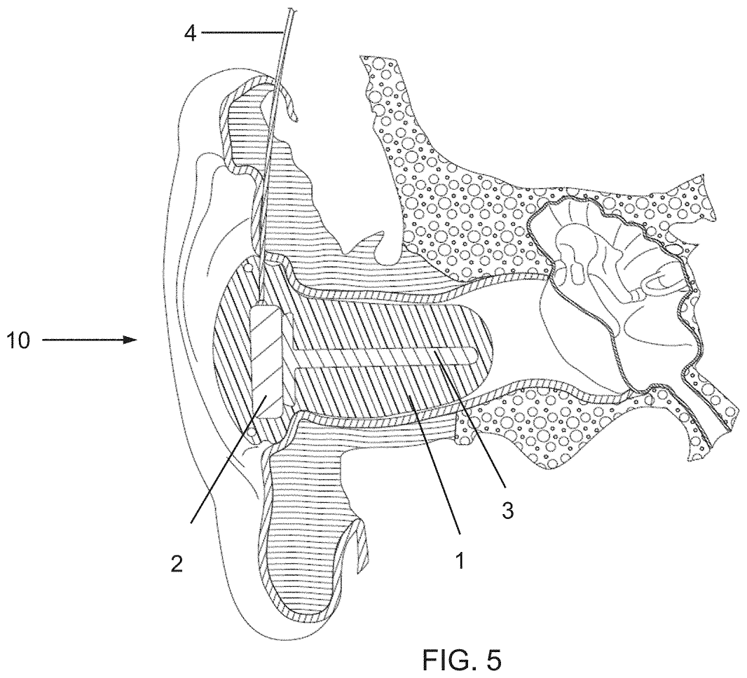

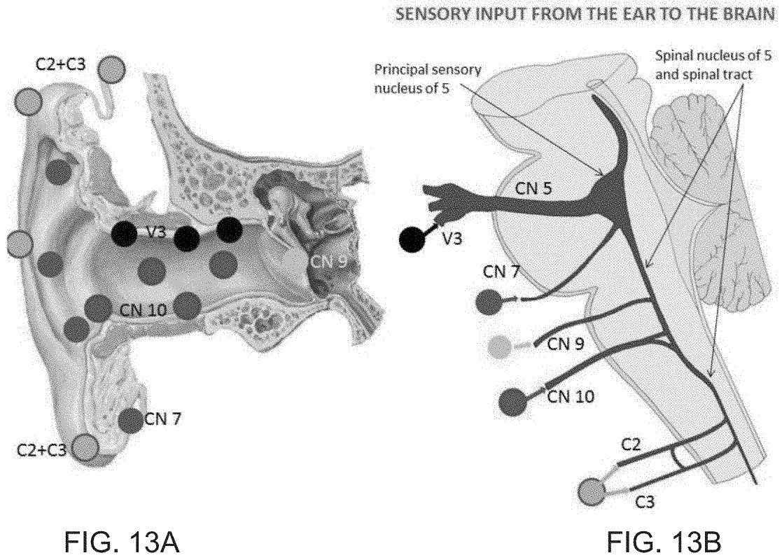

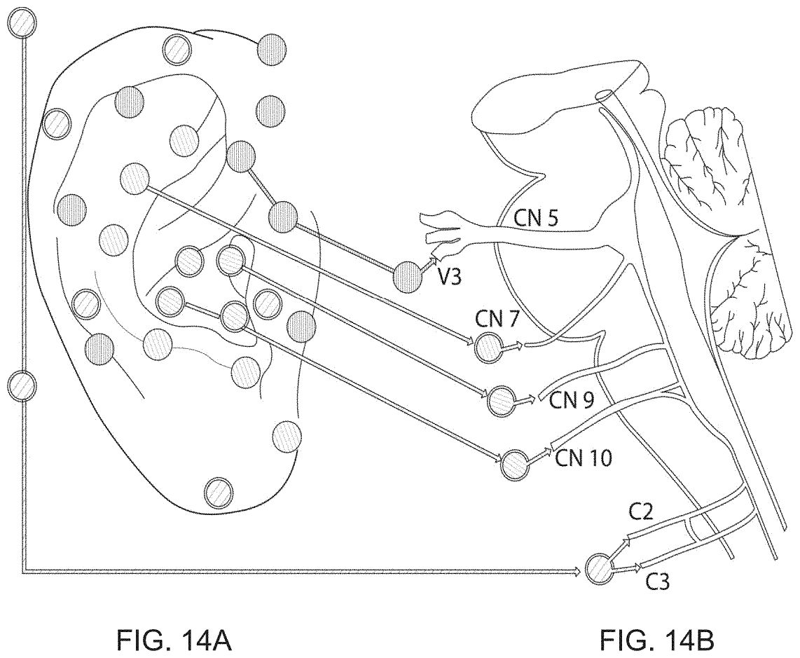

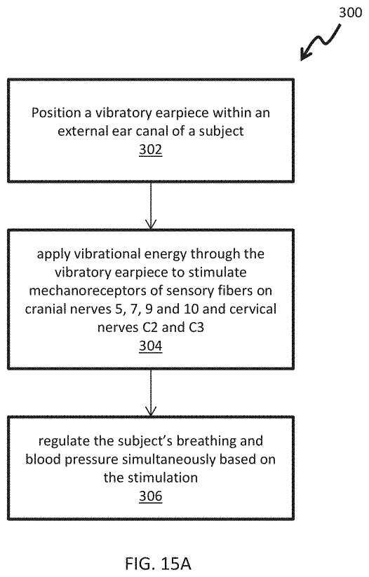

When the vibration motor is activated, vibrational energy is transferred to the metal rod, and subsequently the metal rod conveys the vibrational energy to the wall of the earpiece molding, which is adjacent to, and at least in partial contact with the tissue containing the cranial sensory nerves (FIG. 5 and FIG. 13). Where less-extensive vibration is needed to deeper portions of the auditory canal, the metal rod can be omitted. For example, in certain embodiments, the motor itself is sufficient to deliver the vibrational energy throughout the earpiece. Vibrations are also transmitted through the cartilaginous tissue to the concha and auricle (FIG. 5), recipient of nerve fibers from cranial nerves 5, 7, 9, 10, and spinal nerves C2 and C3 (FIG. 13 and FIG. 14). Activation of the cranial and the two spinal nerves will mask pain perception, principally from cranial nerves 5 and 9, but also from other cranial nerves by projection of the activated nerve signals to the descending spinal nucleus of 5, and to other brain structures (FIG. 12 and FIG. 13).

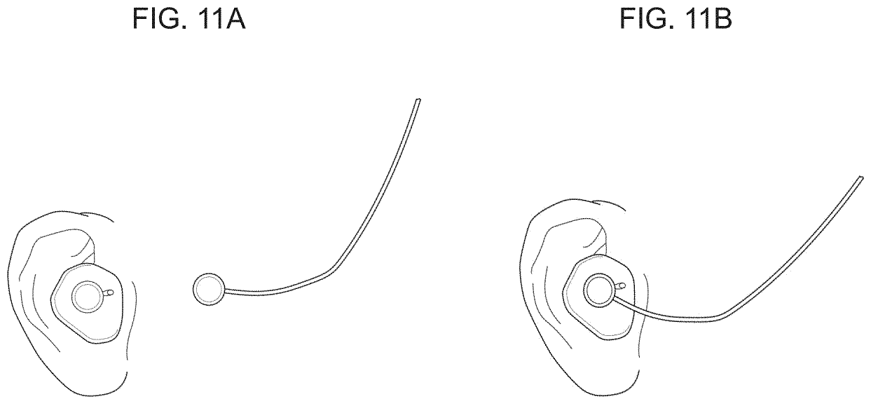

FIG. 11 is a cross-sectional view through the external ear canal and auricle, showing the major structures affected by the device of the present invention. The device preferably reaches the temporal bone just past the junction of the cartilaginous portion of the external ear canal, and may also come in contact with the bony part, and avoid reaching the tympanic membrane (eardrum). The device forms a close seal in the remaining portion of the external auditory canal and concha.

FIG. 12A is an illustration of sensory input from the ear concha and the external ear canal to spinal nerves C2, and C3, and cranial nerves 5, 7, 9 and 10. The external ear canal (external acoustic meatus) is mainly supplied by the third division of cranial nerve 5, cranial nerves 10 and 9; the last serves the area of the tympanic membrane. Vibratory stimuli are transferred from the external ear canal to the tympanic membrane. FIG. 13B is an illustration of the brain stem nuclei involved in sensory information processing of the trigeminal nerve. The principal, or main sensory nucleus of cranial nerve 5 mediates touch, vibration, and pressure, but pain is mediated by the descending or spinal nucleus of 5, which is contiguous with the spinal tract mediating pain from lower nerves. FIG. 12A and FIG. 12B illustrate how pain mediated by cranial nerves 7, 9, 10 and spinal nerves C2 and C3 use the same descending spinal nucleus and spinal tract. FIG. 14A illustrates sensory input from the ear auricle, concha, and the external ear canal to C2, C3, and cranial nerves 5, 7, 9 and 10. The external ear canal (external acoustic meatus) is mainly supplied by cranial nerve 5 (V₃), 7, and 10, and spinal nerves C2 and C3 contribute to the innervation of the auricle. Cranial nerve 9 supplies the area of the tympanic membrane. Vibratory stimuli from the external ear canal are transferred to the tympanic membrane. FIG. 14B illustrates brain stem nuclei as similarly depicted in FIG. 13B.

### Device and System

The device and system of the present invention may be further described in light of and in reference to the following Figures.

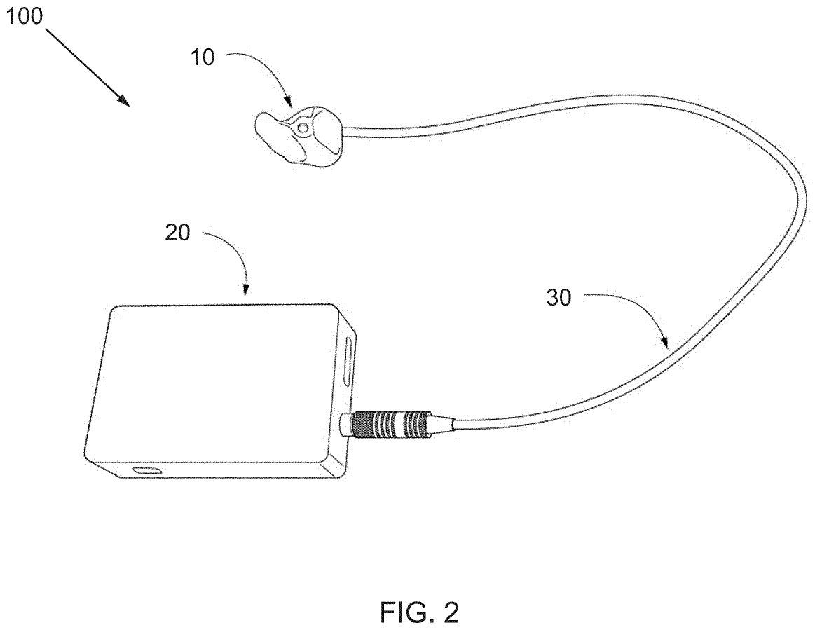

As depicted in FIG. 2, the present invention provides a device 100 comprising an earpiece 10 and control unit 20. Earpiece 10 has a geometry which allows for its insertion into the exterior ear canal(s) of the subject. For example, in certain embodiments, earpiece 10 may be constructed from an impression of the subject's left and/or right ear canals and adjacent concha. Control unit 20 may include the battery power, vibratory stimulation programming electronics, switches to establish settings of stimulus pulse duration, frequency and pattern, and a display screen to indicate appropriate stimulation characteristics. In certain embodiments, control unit 20 comprises one or more output jacks for cables to send and/or receive information to earpiece 10. In certain embodiments, the device comprises two earpieces, each attached to a control unit via cables connected to two output jacks (FIG. 3).

In certain embodiments, control unit 20 is a programmable unit used to deliver electrical pulses to earpiece 10. In certain embodiments, control unit 20 is in wired communication with earpiece 10 via cable 30. In one embodiment, cable 30 is an insulated cable. For example, in one embodiment, control unit 20 is electrically connected to earpiece 10 via a cable or wire 30, as shown in FIG. 2. In other embodiments, earpiece 10 includes a wireless receiver and a power source such that earpiece 10 may wirelessly receive signals from control unit 20 and/or a computing device.

Control unit 20 may be powered by an internal battery, and uses that battery to send stimulation pulses, typically of a voltage of 1.5-5.0 volts. Alternatively, it may include a plug for accessing electricity from a home, hospital or other location providing access to an electrical power source.

In one embodiment, control unit 20 is used to set or alter the amplitude, pulse rate, pulse burst duration, interburst interval and any other parameter of applied vibrational energy, as desired. In one embodiment, vibration may be at a rate of about 20-200 Hz. In another embodiment, the vibration rate may be at about 120-160 Hz. In other embodiments, the rate of pulses and the duration of pulses may be patient- and/or condition-dependent. The total duration of a stimulation session may be established for periods ranging from 1-2 minutes to 60 minutes or more. The bursts of pulses may be set for varying intervals, for example to match the duration and timing of an inspiration or expiration when the device is used for overcoming sleep-disordered breathing. The timing of pulses within bursts can be set to vary between approximately 30-160 Hz for conditions that patients may feel more comfortable.

The amplitude of vibration can be adjusted for effectiveness of intervention and comfort. In one embodiment, the amplitude of voltage to the vibratory motor for may be varied from 1.5 volts to 5 volts by the patient. Control unit 20 may comprise two or more output channels such that two or more stimulation pulses can be outputted as desired.

In one embodiment, control unit 20 can be switch-programmed to vary such characteristics. For example, control unit 20 may comprise one or more depressible buttons, dials, recessed switches or a touch screen through which control unit 20 may be programmed by a user. The application layer of control unit 20 makes certain parameters accessible and modifiable by user. Control unit 20 may include a user interface including a display screen to provide text or other graphics indicating user information, such as pulse parameters of amplitude and intervals, battery power level, and the like.

Earpiece 10 may be placed within the external ear canal, and also contacting the concha for administration of a vibratory stimuli. Vibration may be induced by low-level battery power via an embedded power source or an external control unit, and transmitted to the sensory nerves lining the external auditory canal and concha, and further transmitted to sensory nerves of the auricle through the cartilaginous tissue of the external ear. Activation of the sensory nerves by vibration masks headache pain, which typically arises from the 5^{th}, 7^{th}, 9^{th} and 10^{th} cranial nerves, and C2 and C3 spinal nerves. Vibration can be initiated by the subject, and amplitude and pulse rate stimulation self-varied by the subject to minimize pain and maximize comfort.

In one embodiment, earpiece 10 comprises a housing 1. Housing 1 may be made from any suitable material including, but not limited to, soft or hard silicon-plastic. In one embodiment, housing 1 is a molded housing, where the mold may be derived from an impression the subject's ear. These impressions may then be converted to a custom vibration unit. In certain embodiments, device 100 comprises two separate earpieces 10, one for each of the user's ear. Thus, in one embodiment, housings 1 of earpieces 10 may comprise a molded housing of the user's left ear and a molded housing of the user's right ear. In one embodiment, the impression of the user's ear(s) may include the concha, providing a larger volume for the mold on the ear canal opening. The interior of the mold is filled with silicon-plastic material to effectively carry vibrations to the outer wall of housing 1, allowing stimulation of the surrounding nerves, and carrying vibrations to the cartilaginous tissue and skin of the of the cavity of the concha and adjacent parts of the auricle where cranial nerves 10, 9, 7, 5, and spinal nerves C2 and C3 are represented. Thus, housing 1 of earpiece 10 may be custom-constructed from hard, inert silicone or similar non-tissue irritating material for each subject's ear using conventional earpiece impression procedures understood by those skilled in the art for construction of hearing aids. FIG. 5 depicts an exemplary earpiece 10 positioned in the external auditory canal and concha. As shown in FIG. 5, earpiece 10 does not reach the tympanic membrane (eardrum). Thus, in certain embodiments, the impression used to construct the earpiece mold may use a medially positioned layer of cotton to prevent direct contact with the tympanic membrane. In another embodiment, housing 1 of earpiece 10 may be pre fabricated using standard sizes and geometries of the ear of the mammal (e.g., human, primate, dog, etc.) of which the device is intended for use.

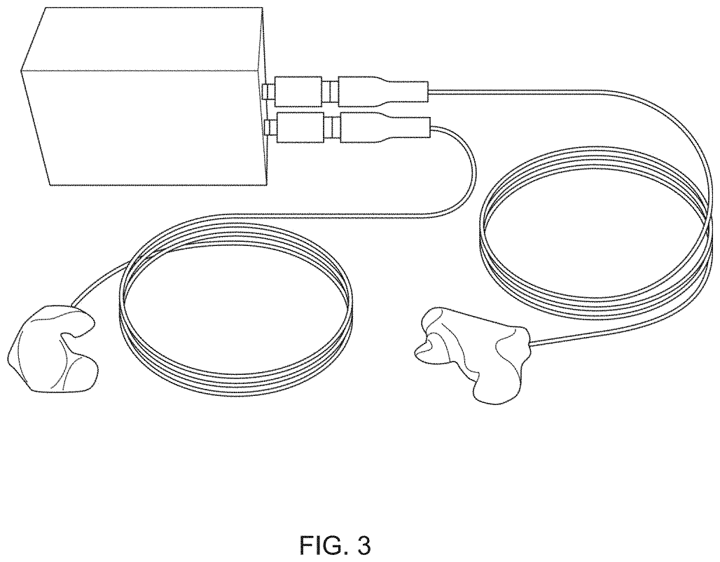

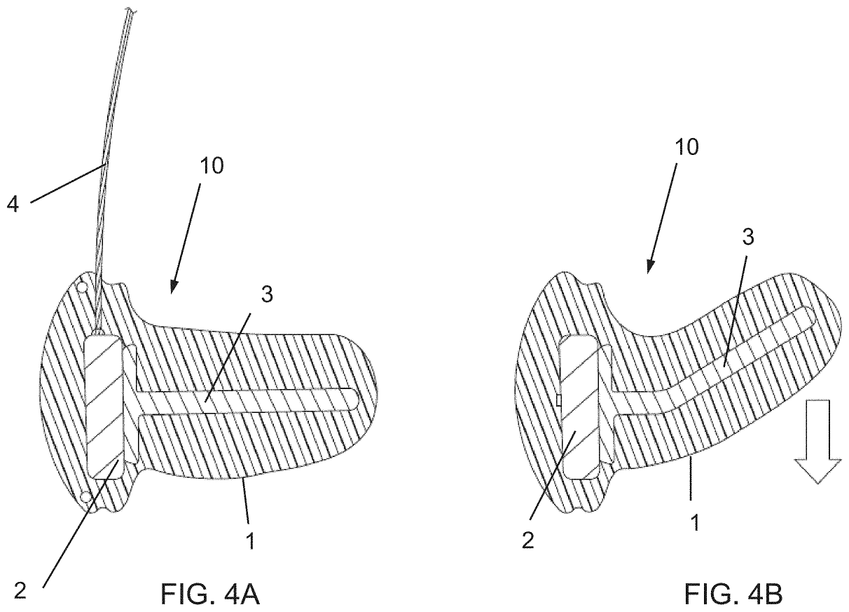

In one embodiment, as illustrated in FIG. 4A and FIG. 4B, earpiece 10 comprises housing 1 within which a vibrating motor 2 is housed. When activated, motor 2 transmits vibrational energy through housing 1 and delivers vibrational energy to the outer wall of housing 1, thereby providing vibratory stimuli to the skin in contact with earpiece 10. Motor 2 may be a small, circular vibrator with a diameter of approximately 2-10 mm. It should be appreciated that vibration motor 2 may be of any type, size or dimension as understood by those skilled in the art, provided the vibrator is capable of neural stimulation, as described herein. For example, in one embodiment, motor 2 is a coin motor.

In one embodiment, vibration motor 2 is housed in an accessible compartment within housing 1. For example, in one embodiment, housing 1 comprises a hinge which allows access to the compartment for replacement of motor 2. In another embodiment, the compartment may be accessed via the removal of one or more screws which allows for removal of a portion of housing 1.

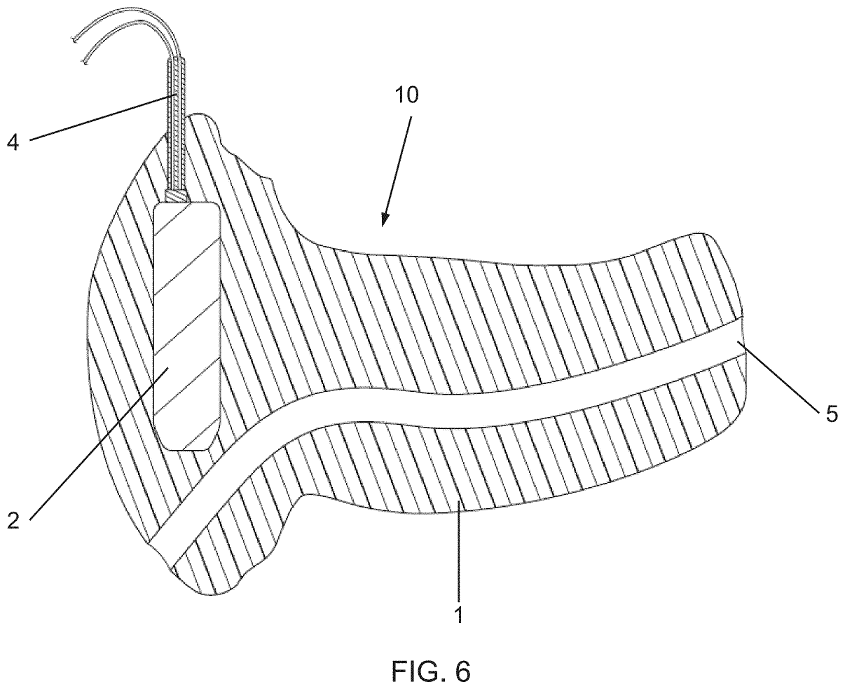

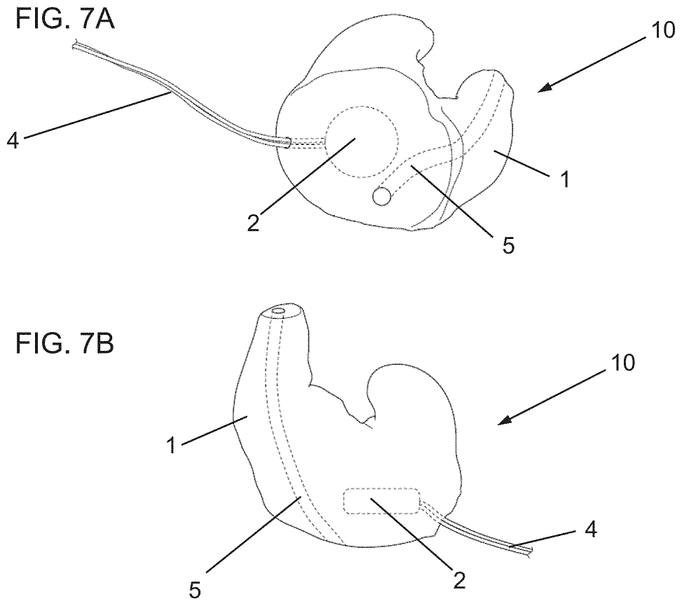

In some embodiments, as shown in FIG. 4A and FIG. 4B, earpiece 10 comprises a metal vibration rod 3, which is attached to vibrating motor 2. Vibrating rod 3 includes a relatively flat head which attaches to or otherwise contacts vibrating motor 2, and an angled rod, with a 35-40° bend in the posterior direction to match the curvature of the ear canal. It should be appreciated that rod 3 may be angled at greater or lesser angles, and of any desired length and diameter, depending on the dimensions most comfortable for placement within any particular subject's ear. Moreover, as described below, and as shown in FIG. 6 and FIG. 7A through FIG. 7B, rod 3 may be eliminated entirely when the solid material of housing 1 needs to convey vibrations principally to less-deep portions of the auditory canal.

In one embodiment, vibration motor 2 is coupled to a wire 4. In certain embodiments, wire 4 is coupled to cable 30, which connects earpiece 10 to control unit 20. In another embodiment, vibrational motor 2 is wirelessly connected to control unit 20 or to an external computing device, as described elsewhere herein.

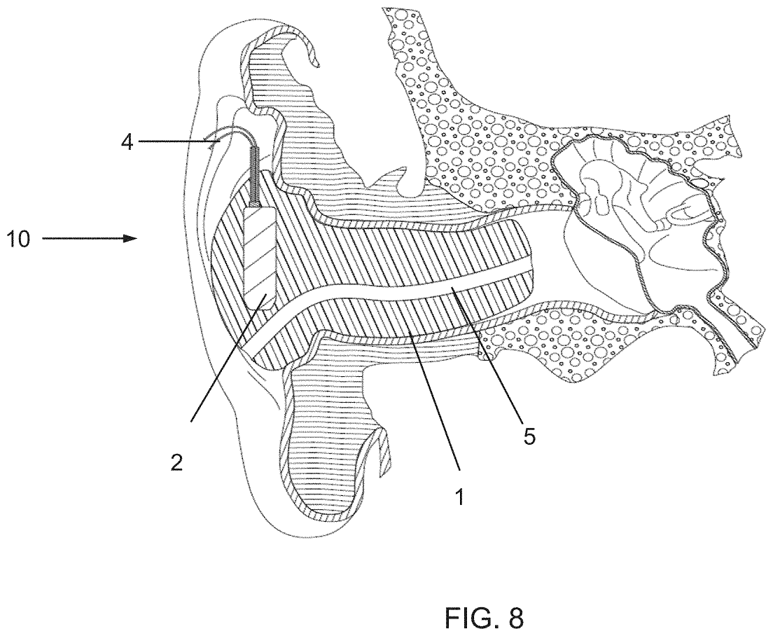

In one embodiment, as illustrated in FIG. 6 through FIG. 8, earpiece 10 comprises a duct 5 which runs through the length of housing 1. Duct 5 allows for the establishment of air pressure equilibrium between the outside air and the ear duct at the tympanic membrane. Duct 5 may be of any suitable geometry and size which allows for the establishment of air pressure equilibrium. Duct 5 comprises an opening at the distal end of the housing, positioned near the tympanic membrane of the ear canal when earpiece 10 is inserted in the ear of the user, and an opening at the proximal end of the housing positioned at the external opening of the ear canal.

In one embodiment, the device is comprised of two separate components, where one component comprises an earpiece containing an embedded and well anchored magnet and the other component comprising a vibratory motor which is fused with a magnet. In certain embodiments, the magnet of the earpiece and magnet of the motor have opposite polarity. For example, in one embodiment, the magnet embedded within the earpiece has a default magnetic polarity of south while the magnet fused to the motor has a default magnetic polarity of north. When the two components are in close proximity, the two magnets automatically latch onto each other and establish a precise and firm connection between the vibrating motor and the earpiece. FIG. 11A and FIG. 11B demonstrates how a subject can connect the two parts of the device without visual control. It should be appreciated that the transmitted vibrations have the same strength and the same physiological effect as the device with the vibrating coin motor directly embedded into the earpiece, described elsewhere herein. In certain instances, this embodiment has several advantages. The separate earpiece that is tailored to a given patient's ear displays enhanced durability. The external and cabled vibratory motor component finds its desired position automatically, but it can be easily removed by sliding action. The vibrating motor component can be easily replaced, and it can also serve any other ear devices.

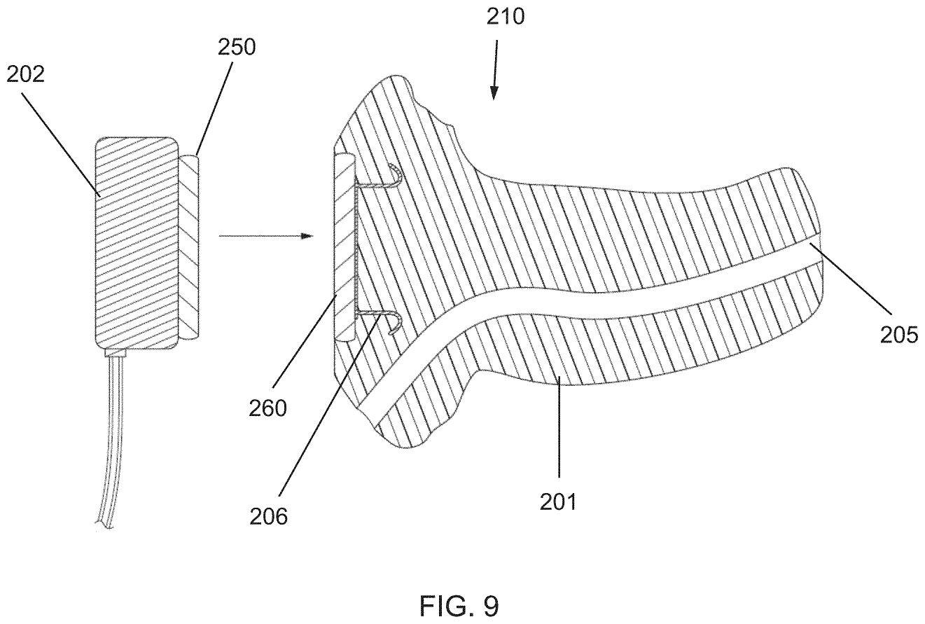

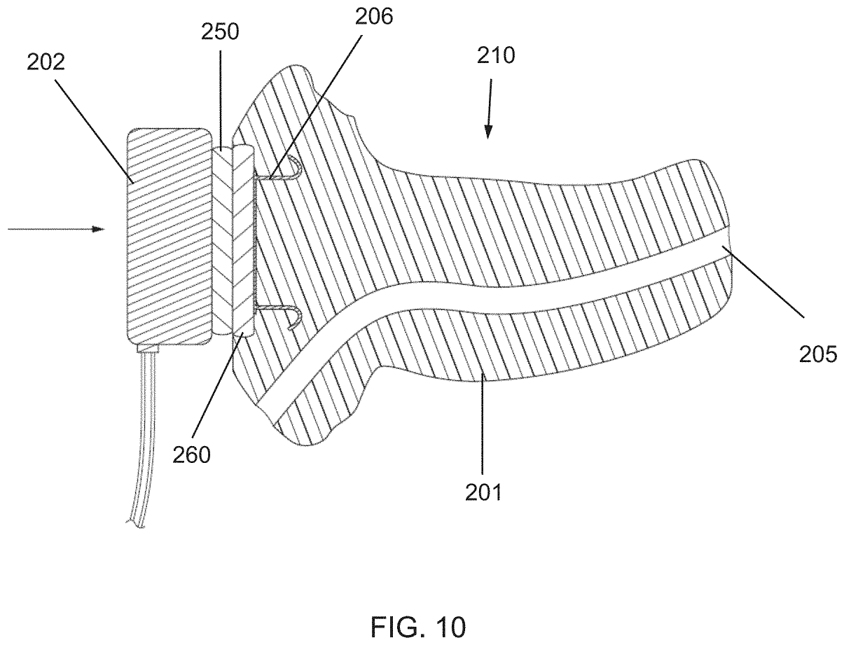

FIG. 9 and FIG. 10 depicts an embodiment of the invention, wherein housing 201 of earpiece 210 is releasably coupled to an external vibration motor 202. For example, in one embodiment, motor 202 comprises a magnet 250, and the proximal end of housing 201 comprises a magnet 260, where magnet 250 and magnet 260 are of opposite polarity, such that motor 202 can be coupled to earpiece 210. Thus, when in proximity, the magnet 250 and magnet 260 attract each other to attach motor 202 to earpiece 210. When activated, motor 202 transmits vibrational energy through housing 201 and delivers vibrational energy to the outer wall of housing 201, thereby providing vibratory stimuli to the skin in contact with earpiece 210. Motor 202 may be detached from earpiece 210 by sliding magnet 250 and magnet 260 relative to each other. Magnet 250 and magnet 260 may be attached to motor 202 and housing 201 using any mechanism known in the art, including but not limited to, adhesives, magnetic anchors, and the like. For example, as shown in FIG. 10, FIG. 11A and FIG. 11B, earpiece 210 comprises an anchor 206 attached to magnet 260 to aid in securing magnet 260 to housing 201. Magnet 250 and magnet 260 may be of any suitable type and size, as known in the art. For example, in one embodiment, magnet 250 and magnet 260 are neodymium disk magnets. Other high-powered types of magnets may be used as understood by those having ordinary skill in the art. In one embodiment, the magnet 250 and magnet 260 have a diameter of about 2-10 mm and a thickness of about 0.1-10 mm. In one embodiment, magnet 250 and magnet 260 exert pull force of about 0.5 to 10 lbs. In certain embodiments, motor 202 is coupled to wire 204 which allows for communication with a control unit and/or a computing device, as described elsewhere herein. Further, in one embodiment, earpiece 210 comprises a duct 205 running through the length of housing 201 to establish air pressure equilibrium. Further, in one embodiment, earpiece 210 comprises a vibrational rod which may aid in transmitting vibrational energy throughout earpiece 210, as described elsewhere herein.

The releasable coupling of motor 202 to earpiece 210 allows for earpiece 210 to remain inserted within the ear of the subject, without being connected to motor 202 or to a control unit, if desired by the subject. Further, as motor 202 is not embedded within housing 201, the size or geometry of motor 202 is not particularly limited to the types of motors which can fit within housing 201. While the present embodiment is exemplified by way of magnetic coupling of the motor, a skilled artisan would recognize that the motor may be releasably coupled to the earpiece housing using alternative methodology, including but not limited to hook and ladder coupling, adhesive coupling, and the like.

In one embodiment, the present invention provides a device and system comprising a computing device in communication with one or more of the control units, earpieces, and/or motors described elsewhere herein. For example, in one embodiment, one or more of the control units are programmed by a computing device, such as a remote desktop, laptop, smartphone, tablet, wearable computing device, and the like, which is in wired or in wireless communication with the control unit. The computing device may comprise software which may establish the amplitude, pulse rate, pulse burst duration, and interburst interval, and any other parameter of applied vibrational energy, as desired. In one embodiment, the computing device outputs a synchronizing signal to store on a recording device when concurrent physiological monitoring (necessary for those subjects who have concurrent autonomic pathology with migraine pain). In certain embodiments, the computing device may be in direct communication, either via wired or wireless communication, with the inserted earpiece and/or the motor attached to or embedded within the earpiece.

In one embodiment, the present invention may be controlled directly by a wireless computing device, such as tablets, smartphones or other wireless digital/cellular devices that are network enabled and include a software application platform or portal providing a user interface as contemplated herein. The applications platform may be a local or remotely executable software platform, or a hosted internet or network program or portal. The computing devices may include at least one processor, standard input, and output devices, as well as all hardware and software typically found on computing devices for storing data and running programs, and for sending and receiving data over a network. The communications network between the computing device and the vibrator component can be a wide area network and may be any suitable networked system understood by those having ordinary skill in the art, such as, for example, an open, wide area network (e.g., the internet), an electronic network, an optical network, a wireless network, personal area networks such as Bluetooth, a physically secure network or virtual private network, and any combinations thereof. In certain embodiments, the computing device comprises a display suitable for visual representation of system control and status. The communications between the computing device and the control unit and/or vibration motor may be conducted via any wireless based technology, including, but not limited to radio signals, near field communication systems, hypersonic signal, infrared systems, cellular signals, GSM, and the like.

In certain embodiments, the computing device comprises a software application used for the input of stimulation parameters, delivery of stimulation parameters, storage of stimulation protocols, storage of user information, and the like. The software application platform may be a local or remotely executable software platform, or a hosted internet or network program or portal.

The software platform includes a graphical user interface (GUI) for inputting stimulation parameters, modulating function of the control unit and vibration motor, and for displaying information regarding the historical or real-time functionality of the device, as well as historical or real-time pain perception. In certain embodiments, wireless communication for information transfer to and from the computing device may be via a wide area network and may form part of any suitable networked system understood by those having ordinary skill in the art for communication of data to additional computing devices, such as, for example, an open, wide area network (e.g., the internet), an electronic network, an optical network, a wireless network, personal area networks such as Bluetooth, a physically secure network or virtual private network, and any combinations thereof. Such an expanded network may also include any intermediate nodes, such as gateways, routers, bridges, internet service provider networks, public-switched telephone networks, proxy servers, firewalls, and the like, such that the network may be suitable for the transmission of information items and other data throughout the system.

As would be understood by those skilled in the art, the computing device may be wirelessly connected to the expanded network through, for example, a wireless modem, wireless router, wireless bridge, and the like. Additionally, the software platform of the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. Access or use restrictions may be implemented for users at any level. Such restrictions may include, for example, the assignment of usernames and passwords that allow the use of the present invention, or the selection of one or more data types that the subservient user is allowed to view or manipulate.

In certain embodiments the network provides for telemetric data transfer to and from the control unit, vibration motor, and computing device. For example, data transfer can be made via any wireless communication technology, including, but not limited to radio signals, near field communication systems, hypersonic signal, infrared systems, cellular signals, GSM, and the like. In some embodiments, data transfer is conducted without the use of a specific network. Rather, in certain embodiments, data are directly transferred to and from the control unit and computing device via systems described above.

The software may include a software framework or architecture that optimizes ease of use of at least one existing software platform, and that may also extend the capabilities of at least one existing software platform. The software provides applications accessible to one or more users (e.g., patient, clinician, etc.) to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system. Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art.

The software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text, or phone call, to alert a patient, doctor, nurse, emergency medical technicians, or other health care provider of the particular results.

### Treatment Methods

The present invention may bring substantial relief of pain to a wide range of headache syndromes, and does so non-invasively and rapidly to the affected person with minimal medical intervention after initial fitting and testing of the device within the subject's external ears. The present invention may reduce pain within seconds of administration, without use of pharmaceutical agents that may have deleterious cognitive, arousal, mood or motoric side effects. Further, the present invention avoids use of electrical stimulation, paralytic muscle agents, such as Botox, or invasive surgery, such as lesions to cranial nerve nuclei to eliminate pain, or vascular decompression surgery to relieve blood vessel pressure from excitable nerves causing pain as are currently used for migraine. The present invention may also be used to “train” brain activity to reduce the incidence of epochs of headache pain, or to minimize the debilitating character of those headache episodes. Such “training” is evidenced by the gradual decline in need to use the method in a group of affected patients; both episodes of pain and incidence of such symptoms as lack of salivation decline with repeated use of the method.

The present invention provides a method of treating or preventing pain in a subject in need thereof, comprising positioning a vibratory earpiece within at least one ear of a subject and applying vibrational energy to at least a portion of the skin of at least one of the auditory canal, auricle and concha of the subject's ear. The method thus stimulates one or more sensory fibers of at least one of cranial nerve 5, cranial nerve 7, cranial nerve 9, and cranial nerve 10, spinal nerve C2, and spinal nerve C3 of the subject.

In some embodiments, a comfortable and effective vibration amplitude and rate level may first be established for the subject by a medical practitioner. Afterwards, the subject may use the device when needed to reduce pain, or, with longer vibration periods, prevent occurrence of epochs of pain. In some embodiments, the subject may report on the efficacy of the device by regularly completing pain scale information. In certain embodiments, a subject being treated, caregiver, and/or medical practitioner may program the vibratory stimulation pattern delivered by the device to best treat the subject. For example, the subject, caregiver, and/or medical practitioner may alter one or more of amplitude, pulse rate, pulse burst duration, interburst interval and any other parameter of applied vibrational energy, as desired. For example, the parameters may be altered based upon observed or reported changes in pain intensity, frequency, duration, and the like.

The present methods may be carried out on any subject. In certain embodiments, the subject is a mammal. In one embodiment, the subject is a human. However, the invention is not limited to use in humans.

The method of the present invention may be used to treat or prevent pain in a subject including, for example pain in the head, neck, oral cavity, or face of the subject. For example, the method may be used to treat or prevent disorders including, but not limited to, primary headache, secondary headache, cluster headaches, migraine, trigeminal neuralgia, glossopharyngeal neuralgia, hemicrania continua, stabbing headache, cough headache, sinus headache, tension headache, exertional headache, sex headache, hypnic headache, cervicogenic headache, radiation pain, burning mouth syndrome, fibromyalgia, and the like. In one embodiment, the method reduces the intensity of pain. In one embodiment, the method reduces the duration of pain. In one embodiment, the method reduces the frequency of the onset of pain. In certain embodiments, the method is used to treat or prevent migraine-associated disorders, including, but not limited to, vertigo, dizziness, hypotension, hypertension, depression, anxiety, bipolar disorder, and the like. In certain embodiments, the method is used to treat or prevent one or more symptoms of migraine or headache pain, including, but not limited to, vision disturbances, altered mood, irritability, fatigue, muscle pain, nasal congestion, constipation, diarrhea, sensitivity to light, sensitivity to smell, sensitivity to touch, hypertension, hypotension, speech disturbances, hallucinations, delusions, weakness, nausea, vomiting, cognitive difficulties, and the like. In certain embodiments, the method reduces one or more of the intensity, duration, or frequency of pain or migraine related symptoms.

In certain embodiments, the method may be used to correct movement disorders of the head and speech.

In certain embodiments, present invention provides a method for induction of sleep. For example, in one embodiment, the method induces quiet sleep followed by rapid eye movement sleep within 15 minutes, and can be used to induce such sleep states.

In one embodiment, the present invention provides a method of treating and preventing xerostomia or dry mouth syndrome. Dry mouth syndrome frequently accompanies neural radiation injury following radiation for oncology, or damage to oral nerves following dental procedures or trauma. Use of the method resulted in gradual increasing of time when salivation was present with repeated interventions by the method, in some cases resulting in remission. The syndrome can result in excessive tooth decay, halitosis, impaired swallowing, and difficulty in chewing and processing certain foods, and greatly interferes with quality of life. Xerostomia treated or prevented by way of the present invention, may occur for a variety of reasons, including but not limited to, dehydration, radiation, side effect of medication, sicca syndrome, Sjogren's syndrome, alcohol use, tobacco use, recreational drug use, diabetes, and the like. It is demonstrated herein that vibrational stimulation of the ear canal of the subject increases salivation in subjects afflicted with xerostomia. Without being bound to any particular theory, the salivation benefit from the present method presumably stems from activation of parasympathetic fibers which accompany branches of cranial nerves 5, 7, and 9, and comprise part of cranial nerve 10. Parasympathetic nerves accompany 5, 7, and 9 on their path to secretory glands, including salivatory glands, and cranial nerve 10 supplies parasympathetic fibers to the pharynx for secretory glands. Vibratory stimulation will cross-activate the accompanying parasympathetic fibers, and also exacerbate neural activation simulating sensory input from the oral cavity that would trigger salivary output.

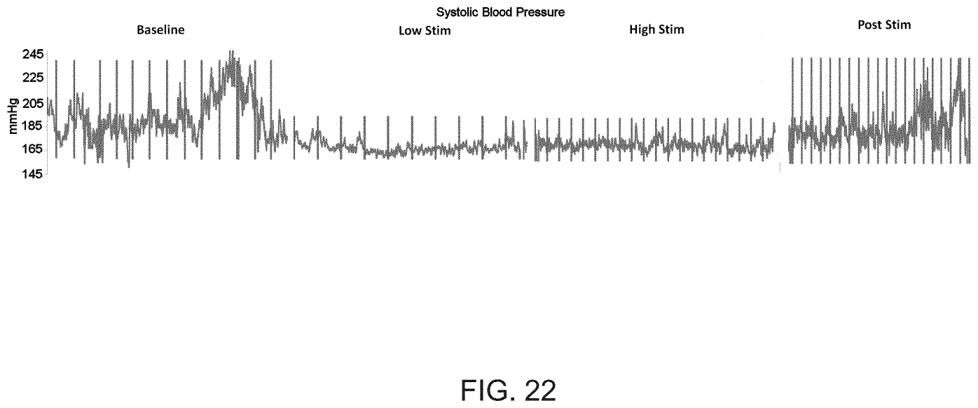

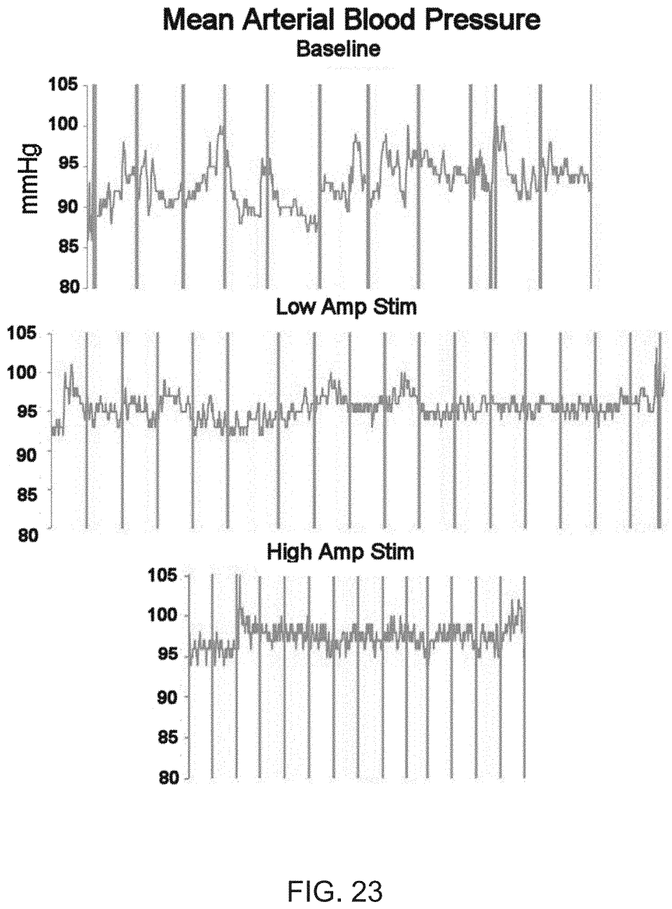

In one embodiment, the present invention provides a method of treating and preventing momentary hypotension in a subject in need thereof. For example, in certain instances stimulation via the device and system of the invention reduces variation in blood pressure and restores proper variation in cardiac R-R interval induced by vagal and 9^{th} cranial nerve processes. The 9th cranial nerve innervates the carotid baroreceptors, which regulate blood pressure and activation of a branch of the 9^{th} nerve can modify that innervation. In addition, cranial nerve 10 innervates the aortic baroreceptors, and carry that information to the nucleus of the solitary tract, a central nervous system responsible for integrating blood pressure. Thus, stimulation of cranial nerve 10 can modify blood pressure sensing by the aortic baroreceptors, and modify activity of the solitary tract nucleus to influence blood pressure. In one embodiment, the present invention provides a method of rapidly lowering excessive high blood pressure by means of stimulation of those two nerves, as is shown in FIG. 22. In another embodiment, stimulation can improve orthostatic hypotension by reducing momentary large variation in, and modestly elevating, blood pressure (FIG. 23).

In another embodiment, stimulation of cranial nerves 5, 7, 9, and 10, and spinal nerve C3 may significantly impact breathing pathologies, including the three most common sources of respiratory deficiencies, namely obstructive sleep apnea, periodic breathing, and hypoventilation, including central apnea (hypoventilation) induced in congenital central hypoventilation syndrome (CCHS), spinal cord injury, or apnea of prematurity.

For example, hypoventilation, the reduced ventilation that occurs in multiple syndromes, results in inadequate flow of air to the lungs from underperforming respiratory musculature, total cessation of all respiratory muscle action (central apnea), or intermittent breathing with breathing pauses (periodic breathing). Accordingly, the device, system and methods of the present invention may be used to treat subjects with conditions involving hypoventilation. The effectiveness of the present invention in such instances stems from the capability to modify activity in nerves that can markedly enhance breathing. For example, motor components of spinal nerve C3 form part of the phrenic nerve output, the principal nerve which drives the diaphragm, and stimulation of C3 sensory components can elicit activity in C3 motor elements. This activation may thus enhance breathing in hypoventilating subjects, a major concern in spinal cord injury during sleep, in some genetic syndromes, such as congenital central hypoventilation, and a range of other pathologies with weakened muscles or impaired neural drive to respiratory muscles. Normalization of breathing patterns in a hypoventilating subject with concurrent obstructive sleep apnea, as described herein, may also be obtained with use of the present invention.

For example, obstructive sleep apnea, the loss of upper airway muscle activity in the presence of continued diaphragmatic movements during sleep, is a syndrome that affects nearly 12 percent of the U.S. population, and results in substantial injurious cardiovascular, memory, cognitive, and blood glucose changes. The objective is to activate the upper airway muscles just before, and during inspiratory diaphragmatic activity, so that the upper airway does not close, as it does in obstructive sleep apnea. Accordingly, the present invention may trigger several sequences that “trick” the brain to activate the upper airway muscles. Stimulation of cranial nerve 10 may provoke sensory activity acting as if that activity was lung inflation, which will normally trigger the upper airway muscles to activate, dilating the upper airway, and preventing airway obstruction. Activation of cranial nerves 5, 7 and 9 also triggers nerve action that is perceived in the brain as airflow, a necessary component to “trick” the brain to stimulate upper airway muscles to avoid upper airway obstruction and maintain breathing.

The present invention may also be used to treat other breathing patterns that do not meet the usual definition of obstructed breathing, periodic breathing, or hypoventilation. For example, Multiple Systems Atrophy, in addition to obstructive sleep apnea and hypoventilation, also shows stridor, a narrowing of the upper airway characterized by unique high-frequency breathing sounds and limited airflow, a characteristic resulting from failed action of the posterior cricoarytenoid (PCA) vocal cord dilators (or hyperactivity of the opposing laryngeal closure muscles). Such failure indicates failure of cranial nerve 10 motor fibers to those muscles. The present invention, by stimulating cranial nerve 10 afferents, may directly enable correction of the failed programming of the upper airway musculature in Multiple Systems Atrophy.

Further, stimulation of cranial nerves 9 and 10, because they provide sensory nerves to the carotid body, a principal sensor for blood pressure (barosensor), may be performed to modify blood pressure; cranial nerve 10, which supplies the aortic nerves and cardiac plexus, can also affect blood pressure regulation. In another embodiment, the presence of afferent nerves from the 9th and 10th cranial nerves allows the present invention to modify signals from the barosensors served by the 9th cranial nerve and the cardiac slowing mediated by the 10th cranial nerve. Activation of the 10th (vagus) nerve, in general, has an anti-arrhythmogenic effect. Several forms of arrhythmia, especially atrial fibrillation found in obstructive sleep apnea, may be modified by stimulation of the 9th and 10th cranial nerves in the external auditory canal by the present invention.

### Non-Invasive Neuromodulation to Regulate Blood Pressure, Respiration, and Autonomic Outflow

Referring now in detail to the drawings, in which like reference numerals indicate like parts or elements throughout the several views, in various embodiments, presented herein are systems and methods for non-invasive neuromodulation.

Neuromodulation, using cranial and cervical nerve mechanical stimulation, can abolish obstructive and central (periodic breathing) events, and concurrently normalize extremes of blood pressure. Cutaneous sensory fields for cranial nerves 5, 7, 9, and 10, and cervical nerves C2 and C3, all lie in the auditory canal or surrounding pinna. Embodiments of the device and method described herein can abolish obstructive and periodic breathing in all subjects, creating a slow, deep, minimally-variant breathing pattern. Blood pressure in individuals with high systolic and diastolic values can be diminished to normative levels, while those with low blood pressure values can benefit from an increase to normative levels. Individual subjects with previously-demonstrated dramatic loss of blood pressure during sleep with modest support of breathing while using CPAP, can have blood pressure restored and oxygen saturations return to near 100%. Both blood pressure and breathing can be supported in sleep-disordered breathing subjects, correcting a blood pressure concern that has not been addressed with conventional devices.

Embodiments of the device use a different approach from proprioceptive stimulation; instead, the device activates sensory fields used by the breathing system to trigger respiratory movements by “tricking the brain” into perceiving that nerves carrying sensations of airflow, upper airway muscle positioning, chemoreception and blood pressure changes, and eliciting reflexive motor actions to activate upper airway and diaphragmatic muscles, as well as ancillary thoracic and abdominal breathing musculature. Embodiments of the device use sensory fields that are distributed within the auditory meatus on one or both sides of the head, and share input with sensory fields of the upper airway (oro-pharynx).

Embodiments of the device and method synchronize sensory information from multiple sources contributing to appropriate timing of airflow and action of respiratory muscles. At the same time, the intervention activates sensory fields of nerves which regulate blood pressure, particularly the nerve serving the baroreceptors, cranial nerve 9. The intervention will thus provide a means to stimulate an area sensitive to multiple sensory nerves serving airway and cardiovascular functions, and do so non-invasively, non-electrically, using an inexpensive vibratory device. The site responsive to multiple sensory processes is the human external ear canal because of its unique multiple sensory nerve innervations. The approach of stimulating the sensory nerves of the auditory canal has been used successfully for reduction of migraine pain in over 60 subjects. That use for pain has been described earlier (PCT Application No. PCT/US14/66191 filed Nov. 18, 2014). The devices can be structured similarly, but vibration parameters for breathing during sleep will differ from those of pain, since long-term vibration will be needed for sleep studies, mandating lower amplitude levels for comfort.

Embodiments of the device, although similar in design to the auditory meatus used for migraine reduction, differ from regional devices described to assist support for breathing and pain reduction (PCT Application No. PCT/US17/32214 filed May 11, 2017). The regional devices use vibratory stimuli, but do so outside the auditory canals, and exert their influences on local fields where relevant nerve fibers innervate the cutaneous surface.

In one embodiment, a method for treating sleep-disturbed breathing includes applying a vibrational stimulus to sensory nerves for simultaneous regulation of blood pressure and respiration. In one embodiment, a system for treating sleep-disturbed breathing includes a vibratory element operationally coupled to a controller, wherein the controller is configured to send a stimulus signal to the vibratory element capable of stimulating sensory nerves for simultaneous regulation of blood pressure and respiration. In one embodiment, a method for maintaining or improving sleep integrity includes applying a vibrational stimulus to sensory nerves for simultaneous regulation of blood pressure and respiration. In one embodiment, a system for maintaining or improving sleep integrity includes a vibratory element operationally coupled to a controller, wherein the controller is configured to send a stimulus signal to the vibratory element capable of stimulating sensory nerves for simultaneous regulation of blood pressure and respiration.

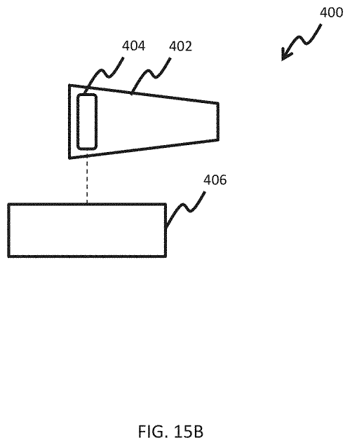

With reference now to FIG. 15A, a method 300 for non-invasive neuromodulation according to one embodiment includes the steps of positioning a vibratory earpiece within an external ear canal of a subject 302; applying vibrational energy through the vibratory earpiece to stimulate mechanoreceptors of sensory fibers on cervical nerves C2 and C3 and cranial nerves 5, 7, 9 and 10 304; and regulating the subject's breathing and blood pressure simultaneously based on the stimulation 306. Since the targeted nerves include autonomic fibers, e.g., 7, 9, and 10, the stimulation can further be used to regulate autonomic outflow based on the stimulating. Mechanoreceptors can be stimulated simultaneously by the vibrational energy. The application of vibrational energy is applied through at least a portion of the skin of at least one of the auditory canal, auricle, and concha of the subject's ear. The stimulation is indicative of nerve sensations for airflow, upper airway muscle positioning, chemoreception, and blood pressure changes. In one embodiment, the stimulation elicits reflexive motor actions to activate upper airway muscles, diaphragmatic muscles, ancillary thoracic musculature, and abdominal breathing musculature. The stimulation reduces blood pressure of the subject by regulating blood pressure, increases blood pressure of other subjects with low blood pressure by regulating blood pressure, enhances breathing of the subject, and regulates autonomic outflow of the subject. The method can treat autonomic disorders and sleep-disturbed breathing. A device 400 for non-invasive neuromodulation is shown with reference to FIG. 15B according to one embodiment, including an earpiece 402 comprising a housing molded substantially to fit within the external ear canal a subject, a vibratory motor 404 connected to the housing, wherein the vibratory motor transmits vibrational energy to an outer wall of the housing, and a controller 406 configured to generate a stimulation signal for stimulating one or more mechanoreceptors of sensory fibers of cranial nerves 5, 7, 9 and 10 when the housing is positioned in the external ear canal for simultaneously regulating the subject's breathing and blood pressure. The vibratory motor 404 can be connected, embedded, or releasably connected to the earpiece 402. The controller generates a stimulation signal that can be configured to regulate autonomic outflow.

The stimulation signal can be configured to stimulate mechanoreceptors simultaneously, reduce blood pressure of the subject for regulating blood pressure, increase blood pressure of the subject with initial low blood pressure for regulating blood pressure, enhance breathing of the subject, regulate autonomic outflow of the subject, and treat autonomic disorders or sleep-disturbed breathing.

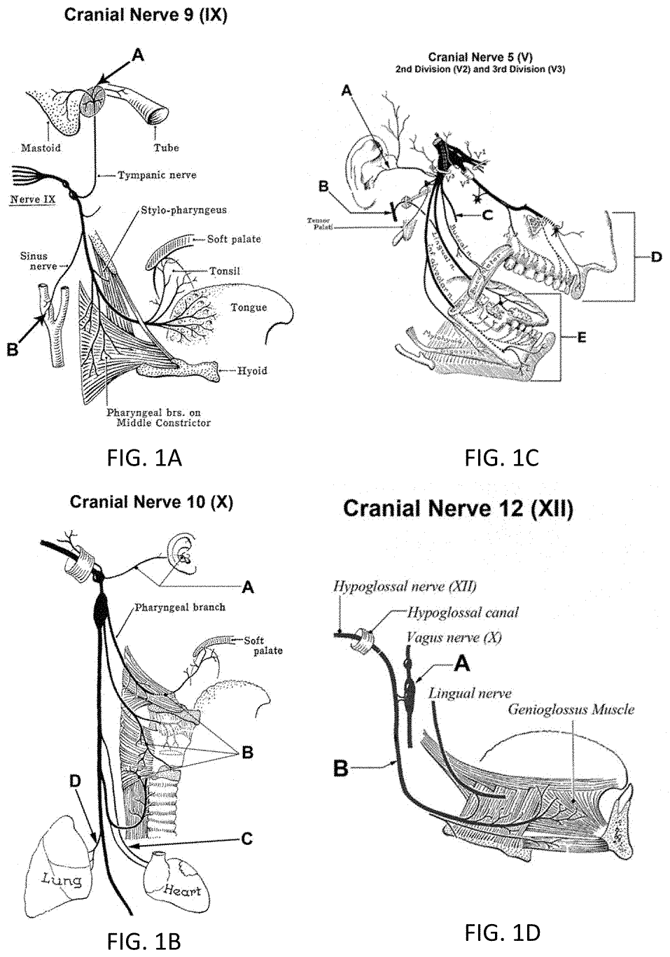

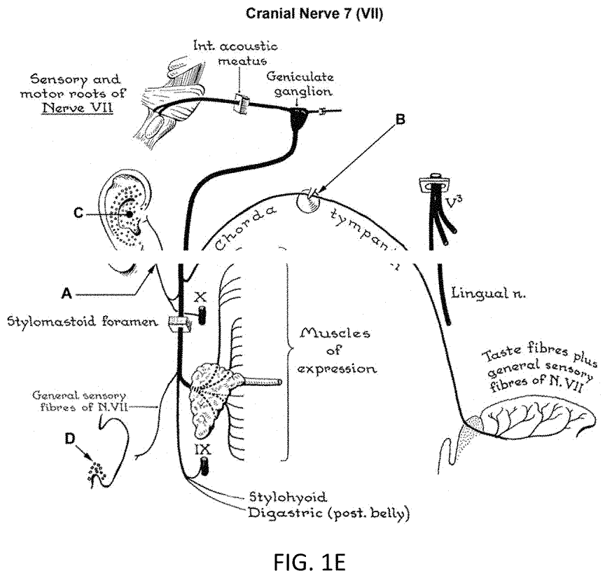

Vibrations within the external ear canal (external acoustic meatus) reach and affect the sensory input to a remarkable number of cranial nerves which also serve sensory attributes of air passages in breathing and sensory signals to baroreceptors and other cardiovascular receptors (FIG. 1A).

The respiratory-related nerves, in turn, all provide background tone to brain regulatory processes driving breathing activation, and also serve respiratory timing roles in breathing regulatory areas of the brain stem. The latter role is critical to, for example, dilating the upper airway before diaphragm descent, thus preventing airway obstruction which occurs if the diaphragm creates negative pressure with a flaccid upper airway. The sensory fields of cranial nerve 9 provide signaling to adequately time changes in blood pressure with respiratory patterning alterations, an essential issue during obstructive sleep apnea (associated with major blood pressure changes) and periodic breathing (accompanied by significant declines in perfusion). In some conditions, such as congenital central hypoventilation syndrome, in which subjects have complete and sustained cessation of breathing during sleep (central apnea), the blood pressure signaling by the 9^{th} nerve (as well as cranial nerves 5, 7, and 10) are even more essential, since the ancillary signaling to blood pressure regulatory areas from respiratory receptor fields has disappeared, leaving 9, 10, 7, and 5 nerve activity alone to support blood pressure.

The external acoustic meatus (approximate length in the adult: 2-3 cm) is primarily innervated by sensory fibers of the trigeminal nerve (cranial nerve 5; specifically, the mandibular division) and the vagus nerve (10); other fibers of those cranial nerves serve roles in sensing air flow, proprioception, lung expansion, and blood pressure regulation.

Vibrations also reach the tympanic membrane. Its external surface is innervated by a branch of the auriculotemporal nerve (CN 5); its internal surface is innervated by the glossopharyngeal nerve (9). The third division of 5 serves oral airflow and proprioceptive roles, and 9 serves essential roles in O₂ and CO₂ sensing, as well as blood pressure sensing. In addition, CN 9 carries vital air movement and other general sensory information from the posterior oral cavity. Cranial nerve 7 has the potential to carry oral airway flow and proprioceptive sensations via the chorda tympani; demonstration of the role of the 7^{th} nerve role in innervation of the tragus of the ear can be shown in pathological cases such as herpes zoster (FIG. 1E).

Thus, non-invasive, vibratory stimulation of the external ear canal will excite multiple and massive sensory input of the following cranial nerves: trigeminal (5), facial (7), glossopharyngeal (9), and vagus (10); these nerves are all involved in respiratory timing and ventilatory extent, and all contribute to blood pressure regulation. They have been previously shown to induce sleep, reduce migraine pain, vestibular migraine symptoms, respiratory rate, and variability, and normalize systolic and diastolic blood pressure in hypertensive cases (Feulner, L C et al., 2017, Am J Respir Crit Care Med., 195:A2570; Harper, R M et al., 2016, Society for Neuroscience Abstracts, 145.20; Harper, R M et al., 2019, International Society for Autonomic Neuroscience, 1 lth Congress, Los Angeles. ISAN 19.117; Harper, R M et al., 2017, Clinical Autonomic Research 27: 295; Harper, R M et al., 2016, ISAN, San Diego).

Connections between the respective sensory and motor nuclei within the brain stem are well-known, and form the basis for knowledge of stimulation of cardiovascular and respiratory control mechanisms. The vagus (cranial nerve 10) projects to pontine respiratory phase-switching and blood pressure regulation sites, as well as to the cardiorespiratory integrative site of the nucleus tractus solitarius (NTS), the glossopharyngeal (9) nerve projects to the NTS and receives baroreceptor information from cells of the carotid body, a portion of the facial nerve (7) projects to the NTS, and the trigeminal nerve projects to the mesencephalic proprioceptive nucleus of 5, integrating muscle coordination of the upper airway, as well as to the motor nucleus of 5, serving oral musculature. A key aspect is that cranial nerve 5 also projects to the NTS with essential cardiovascular roles.

A diagram of the device in place, with attached vibrator motor is shown below:

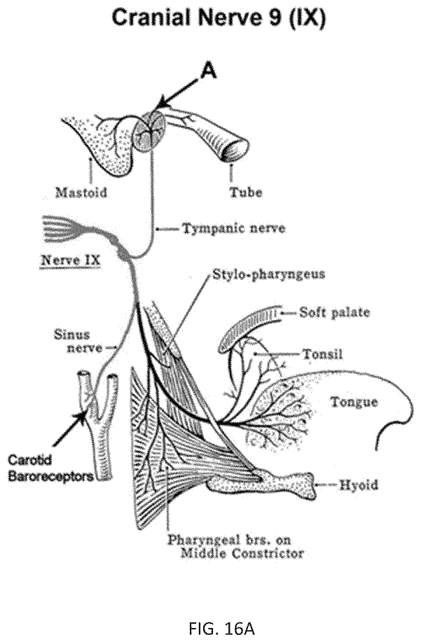

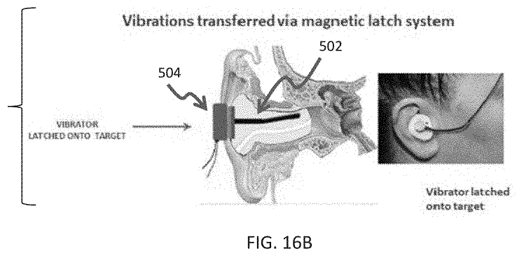

With reference to FIG. 16B, a diagram of silicon impression 502 in one auditory meatus with embedded metal probe to carry vibrations from a vibratory motor 504, which is powered through electrical wires from a remote 3 volt power supply. The metal probe often has a bend, since the auditory canal often follows a curved path sometimes nearly at right angles, from the external surface to the tympanic membrane, and the bar must follow that path. A silicon impression and vibratory motor in situ of a patient is shown to the right.

### Process:

The device to stimulate sensory nerves assisting breathing patterns and cardiovascular regulation is a vibratory device placed in the auditory meatus; various configurations of the vibratory device exist. The most useful is a configuration which contains a fixed magnetic disc cemented to a metallic bar which extends within the silicon impression, nearing the tip. A vibratory latch cable, with a vibratory motor and attached magnet at one end latches to the magnetic disc of the silicon impression. The vibratory motors are attached to leads which provide DC power from the power supply. In another configuration, the vibratory motor can also be placed directly in the silicon impression and driven by directly coupled leads from a separate container with a battery supply and electronic circuitry. Three variations of vibratory stimulation may prove useful; 1) a variable-frequency continuous stimulation (stimulation similar to that shown to be effective with peripheral limb movement in premature infants and in congenital central hypoventilation syndrome); 2) a burst sequence of vibration timed to be slightly advanced over the subjects' diaphragmatic action, and lasting for the duration of inspiration; 3) a burst of vibration at an effective respiratory interval, but not linked to diaphragmatic action, and lasting typically for an inspiratory duration. For cardiovascular regulation, sustained, uniform vibration at a near constant frequency has been found useful.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

In an experimental example, pain perception was examined in 18 subjects with chronic migraine or trigeminal pain which was inadequately controlled by current medications. A plastic earpiece containing a vibrating element was placed within the external ear canal of each subject. Vibration induced by low-level battery power from a remote stimulation device was initiated by each subject after 1 min of onset of pain, and continued for a period of 20-50 minutes. Ratings of pain severity were made by the patient, using an appropriate pain assessment scale. A second, but closely related study examined the incidence of migraine episodes in subjects who have repetitive and quantifiable onsets of migraine. These subjects received vibratory stimuli for 20-50 when they begin to experience migraine pain. The change in perceived pain, as well as the time between episodes of migraine pain was tabulated.

### Subjects

Eighteen adult subjects, aged 19-76 years or age, diagnosed with moderate-to-severe migraine by a UCLA headache pain physician were recruited. Subjects were in otherwise good health, with representation by sex in the same ratio as condition representation, i.e., 4 males and 14 females. Race, sex, or national origin were not reasons for exclusion. Subjects with cardiovascular, especially those with cardiac arrhythmia, or major psychiatric disorders were excluded. Subjects were recruited by notices in the UCLA Headache Clinic.

### Earpiece

Subjects had a custom earpieces constructed from inert silicone using conventional earpiece impression procedures, as commonly employed for hearing aids. The custom earpieces were constructed with sufficient safeguards, using cotton or other soft material at the medial portion of the external ear canal so that no injury to the tympanic membrane occurs, while still ensuring appropriate contact for stimulation at appropriate sites in the canal. The impressions were taken by an audiologist experienced in such procedures at a time prior to experimentation, with the procedure requiring approximately 20 minutes. The impression was converted to a positive mold containing the vibration device, and the subject returned for the experimental session.

### Vibration Device

The vibration device consists of two components: 1) the custom earpiece, derived from an impression of the subject's left and right ears; these earpieces contain the vibration motors attached to a small metal rod which contacts the inner wall of the silicone mold on an area proximal to the receptive field on the ear canal tissue, and 2) a programmed stimulation device (control unit) containing a low voltage battery which powers the vibration motors. The vibration motor is similar to those found in powered toothbrushes or cell phones. The stimulation device is programmed via Bluetooth signals from an Android tablet or smartphone.

### Autonomic and Respiratory Monitoring

Migraine episodes can be accompanied by aberrant autonomic patterns, such as nausea and cardiovascular changes. Any intervention which may modify migraine episodes may also alter autonomic aspects, and thus, these autonomic characteristics need to be evaluated during such interventions. Prior to any assessment of pain or vibration characteristics, the subject was seated in a comfortable chair, and instrumented with a thoracic pressure band to assess thoracic wall movements; ECG electrodes on the medial thoracic wall near the heart, on the lateral thoracic wall opposite the first electrode, and at the caudal end of the sternum; and a pulse oximeter on the index finger. Leads from those electrodes were connected to a SOMNOtouch RESP device (SOMNOmedics, Coral Gables, Fla.) for collection of ECG, thoracic wall movements, oxygen saturation, pulse transit time, and beat-by-beat systolic and diastolic blood pressure (determined from pulse transit time). Conventional cuff blood pressure measurements was determined on the subject prior to, and at the end of data collection, to calibrate the SOMNOmedics blood pressure device. The device allows continuous monitoring of signals on a small display screen so that abnormal variation can be readily observed. All data was subsequently transferred from the device to computing device.

### Pain Scale

The Numerical Rating Score for Pain (NRS) was completed by the subject at onset of the first trial, when the patient reports that he/she is undergoing a moderate-to-severe headache, and at the end of the experimental session for the day. This scale is a uni-dimensional single item scale that provides an easy-to-administer and score scale that allows subjects to rate pain from 0-10 in intensity, and is widely used in the pain field (Hawker, G. A. Mian, S., Kendzerska, T., French, M. Measures of adult pain. Arthritis Care and Research 2011; 63:5240-S252). It requires about 1-2 minutes to administer.

The following pain scale (1-10) is used to classify pain from the subjects:

1: Very mild=Very light, barely noticeable pain, like a mosquito bite or a poison ivy itch. Most of the time you never think about the pain.

2: Uncomfortable=Minor pain, like lightly pinching the fold of skin between the thumb and first finger with the other hand, using the fingernails.

3: Tolerable=Very noticeable pain, like an accidental cut, a blow to the nose causing a bloody nose, or a doctor giving you a shot. The pain isn't so strong that you can't get used to it.

4: Distressing=Strong, deep pain, like an average toothache, the initial pain from a bee sting, or minor trauma like stubbing your toe real hard. So strong that you notice the pain all the time and can't completely adapt.

5: Very distressing=Strong, deep, piercing pain, such as a sprained ankle when you stand on it wrong, or mild back pain. Not only do you notice the pain all the time, you are now so preoccupied with managing it that your normal lifestyle is curtailed.

6: Intense=Strong, deep, piercing pain, so strong that it seems to partially dominate your senses, causing you to think somewhat unclearly. Comparable to a bad non-migraine headache combined with several bee stings or a bad back pain.

7: Very intense=Same as 6, except that the pain completely dominates your senses causing you to think unclearly about half the time.

8: Utterly horrible=Pain so intense that you can no longer think clearly at all, and have often undergone severe personality change if the pain has been present for a long time. Comparable to childbirth or a real bad migraine.

9: Excruciating unbearable=Pain so intense that you can't tolerate it and demand pain killers or surgery, no matter what the side effects or risk.

10: Unimaginable unspeakable=Pain so intense that you will go unconscious shortly.

### Acute Vibration

Following completion of the pain rating, the earpiece was inserted, and the subject questioned on comfort of the device without vibration. Following affirmation of comfort, a 120-160 Hz vibratory signal was applied, initially at low amplitude, and upon confirmation of comfort levels, with increasing levels until the subject reports mild discomfort from the local vibration. The amplitude was then lowered to a level congruent with the subject reports of a comfortable setting. Typically, trials begin with a low amplitude level (1.5 volts) for 10 minutes, after which the trial continues with a higher amplitude (3.0 volt) signal. Before, and at the end of the trial, the pain scale is administered. The physiological electrodes and the earpiece is then removed. The subject is queried on general pain perceptions, any other affective perceptions, and preferences on whether they would like to use the device again.

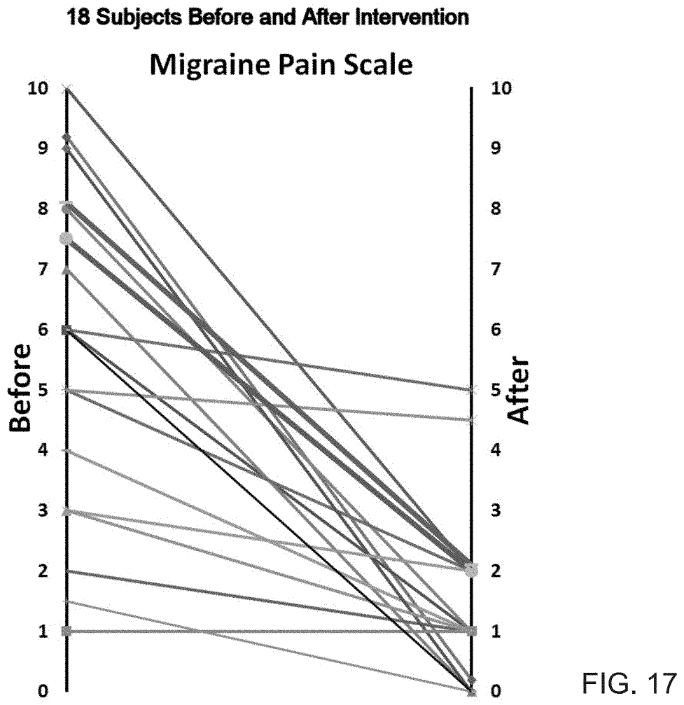

The outcomes of the pain ratings for all 18 subjects are shown in FIG. 17. Of the 18 subjects, 17 showed reduction in reported pain, and one showed no change. In some cases, the pain reduction was substantial (e.g., on a pain scale of 0 to 10, where a rating of 10 is so severe that the subject is at risk to go unconscious, 9 to 0 on two subjects, 10 to 2 on one subject, 8 to 0, 8 to 1, and 8 to 2 on three additional subjects). That outcome is statistically significant (p<0.0001, paired t-test).

### Example 2: Severe Migraine and Trigeminal Neuropathy

A 38 year old female subject with severe migraine and orthostatic hypotension, with a secondary diagnosis of post-traumatic stress syndrome for approximately 3 years was treated using the device of the present invention. The subject's pain had been poorly controlled by opiates and antidepressants.

The parameters of stimulation were as with all 18 subjects; an initial baseline with no stimulation, a 10 minute low amplitude (1.5 volt, 120 Hz vibration), followed by a 20 minute high amplitude (3.0 volts) vibration, and a 5 minute post stimulation baseline.

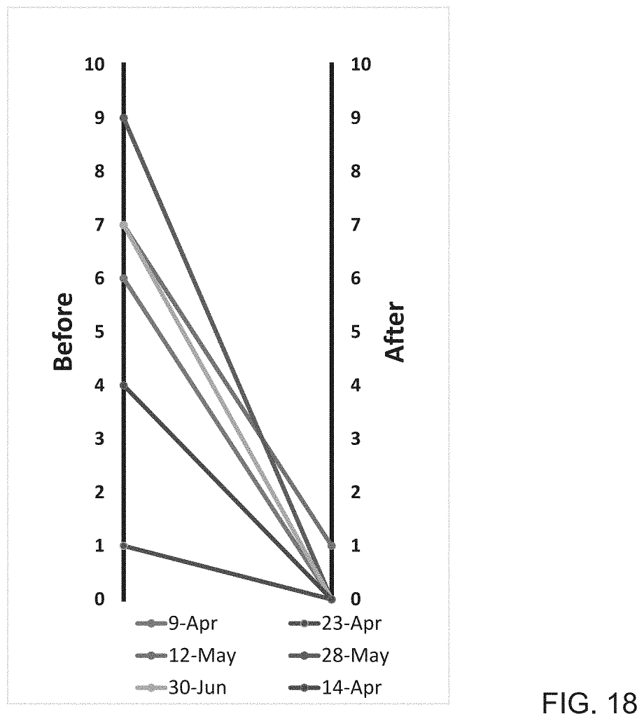

The subject received vibratory stimulation of the ear canal using the present device on 6 separate treatment sessions. As shown in FIG. 18, reported pain decreased at each session, with pain reported at 0 or 1 after each session.

A 19 year old female subject with severe migraine for approximately 2 years was treated using the device of the present invention. The subject's pain had been uncontrolled by medication.

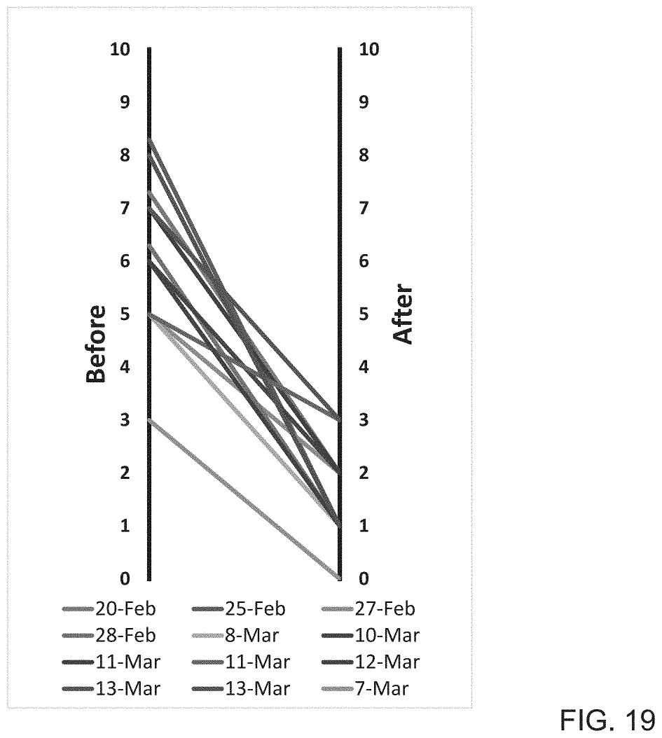

The subject received vibratory stimulation of the ear canal using the present device on 12 separate treatment sessions, using parameters of 5 minute baseline with no stimulation, 10 minutes of low amplitude (1.5 volt) 120 Hz stimulation, followed by 20 minutes of high amplitude (3.0 volts), and a subsequent no stimulation post baseline. As shown in FIG. 19, reported pain decreased at each session.

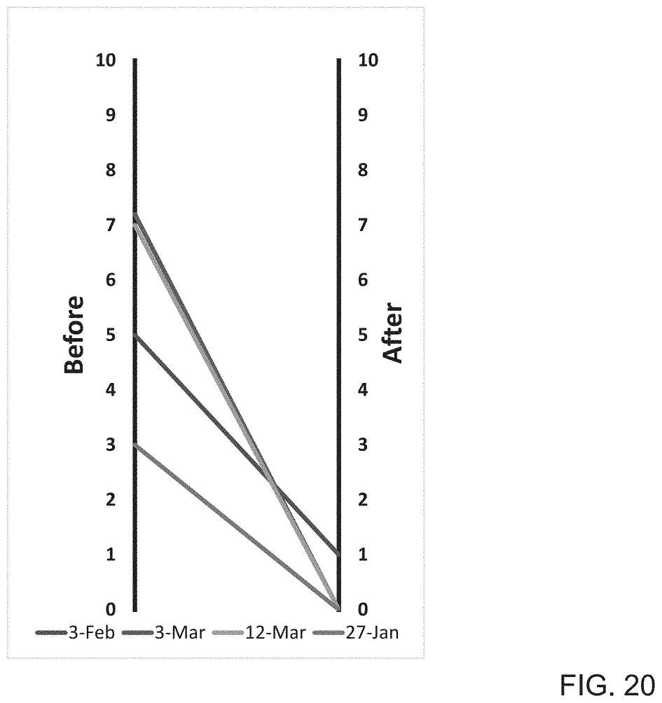

A 48 year old male subject with trigeminal neuropathy for approximately 5 years was treated using the device of the present invention. The trigeminal neuropathy of the subject stemmed from a root canal procedure. The subject's pain had been poorly controlled by Tegretol (carbamazepine). The subject received 10 minutes of low amplitude (1.5 volts to the motor) vibratory stimulation (120 Hz) of both ear canals followed by high amplitude (3.0 volts to the motor) vibration for 20 minutes using the present device on 4 separate treatment sessions. As shown in FIG. 20, reported pain decreased at each session, with pain reported at 0 or 1 after each session. In addition, profuse salivation and tearing occurred, a great benefit for the patient who also suffered from dry mouth.

FIG. 17 depicts the results of vibratory stimulation of 18 different subjects with migraine and/or trigeminal neuropathy. It is demonstrated that the pain decreased in 17 subjects after stimulation, with pain remaining unchanged in only one subject.

### Example 3: Burning Mouth Syndrome

A 73 year old female subject with burning mouth syndrome, characterized by burning sensation of the tongue, pain in the upper and lower mucosa of the anterior oral cavity, and paresthesia in the lips, for approximately 2.5 years was treated using the device of the present invention. The subject's pain had been poorly controlled by Gabapentine.

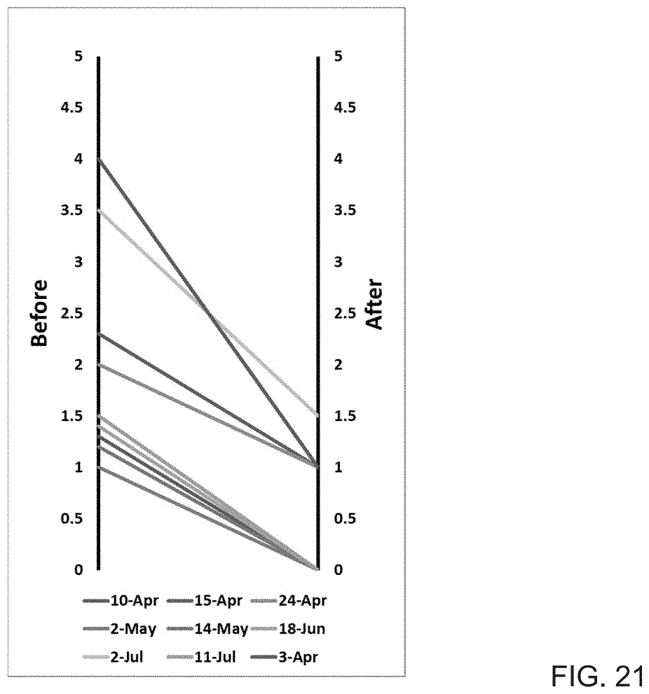

The subject received 120 Hz vibratory stimulation of both ear canals using the present device on 9 separate treatment sessions. Stimulation was comprised of an initial period of 10 minutes of low amplitude (1.5 volt to the motor), followed by high stimulation (3.0 volts of the motor). As shown in FIG. 21, the reported pain decreased at each session, with longer intervals between sessions, beginning initially with four days before re-intervention was required, extending to 2-3 weeks between necessary interventions; pain is now entirely gone, and she no longer returns for sessions. In addition, salivation increased following each intervention.

### Example 4: Hypotension

For safety reasons, all pain interventions were accompanied by simultaneous recording of heart rate, thoracic and abdominal breathing movements, oxygen saturation, and beat-by-beat blood pressure. These recordings provided the serendipitous findings of long-term and momentary changes in breathing, blood pressure and cardiac variability (R-R intervals) associated with stimulation.

The findings indicate a substantial reduction in wide swings in blood pressure in a 38 year old female orthostatic hypotensive patient (being treated for migraine), to relatively stable variation, with an overall restoration to normotensive levels (FIG. 23). In addition, blood pressure returned to normal patterns of variation with breathing. The patient required use of a wheelchair, since turning of the head, or arising from a sitting position, resulted in syncope. Those fainting episodes were abolished by the third session; subsequently the patient abandoned wheelchair use.

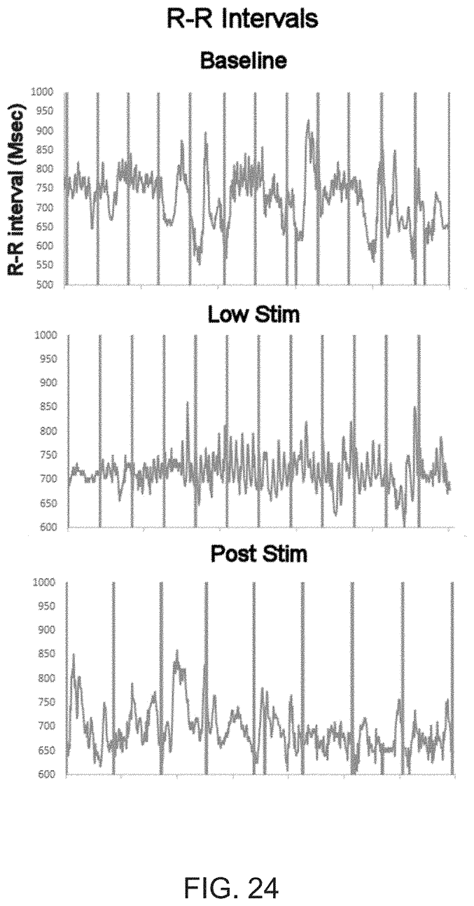

Cardiac R-R intervals, which showed wide variation during baseline pre-stimulation, showed a return to pronounced respiratory-related variation, i.e., marked high frequency variation, a pattern considered in the cardiovascular field as a vagally-mediated cardioprotective pattern (FIG. 24).

### Example 5: Hypertension

A 72 year old male patient with dangerously high blood pressure and concurrent migraine pain underwent one session of stimulation. Systolic arterial pressure ranged up to 240 mmHg during baseline (FIG. 22), but rapidly declined (within 4 minutes; 4 vertical red lines on FIG. 22) during low amplitude (1.5 volt) stimulation to values near 150 mmHg, and remained at these lower levels during high amplitude (3 volt) stimulation; on cessation of all stimulation, systolic pressure rose again to values near 245 mmHg. Continuous vibration at 120 Hz was applied, with no variation in pulse characteristics, except for amplitude change from 1.5 to 3 volts for low (10 minutes of low amplitude) and 20 minutes of high stimulation. Migraine pain was reduced from an initial level 3 to 0.

### Example 6: Salivation

A frequent accompaniment of trigeminal neuropathy is poor salivation, or xerostomia (dry mouth syndrome), and frequently occurs after radiation treatment for oral oncology, or after trauma to the trigeminal or 7th cranial nerve. The condition leads to enhanced tooth decay, and greatly impairs quality of life, since eating particular foods, swallowing, and taste are greatly affected. The condition also tends to elicit reflexive tic-like oral movements, such as lip smacking to overcome dryness. The subjects underwent the usual stimulation parameters of no stimulation during a 10 minute baseline, 10 minutes of 120 Hz vibration at low (1.5 volt) level, 20-30 minutes of high (3.0 volt) level, and a short post baseline. Seven of the 18 patients reported the dry mouth condition, one from radiation treatment, and another from cervical nerve trauma, and the remaining from other oral nerve trauma; two had reflexive lip smacking movements (common in dry mouth syndrome to maintain hydration). All reported improvement, and in some cases, substantial improvement, which included partial resolution of the reflexive lip motor signs. The condition is difficult to quantitatively measure, but one subject who used Pilocarpine, an agent to enhance saliva, reported that she was able to avoid use of the agent initially for one day, and with successive interventions for 2-3 days, and most recently, for 4 days. Another patient reported reduction of the dry mouth for one week after the initial intervention, which corresponded to reduction in burning mouth pain over the same time period. Another patient, who received radiation therapy for cancer of the parotid, had severe migraine, dry mouth, and impaired speech. Pain diminished, salivation increased, and the subject's speech became more articulate.

### Example 7. Sleep Induction

Twelve of the 18 subjects fell into quiet asleep during the stimulation; sleep onset occurred as early as 12 minutes following onset of stimulation. The stimulation parameters were the usual parameters of 120 Hz vibration at 1.5 volt (low level) and 3.0 volt (high level) for 10 and 20-30 minutes, respectively. The sleep events all appeared during interventions carried out in the daytime, at times when the subjects would not normally sleep. On three occasions, the person accompanying the subject expressed amazement that sleep was occurring, given the subjects' usual behavior at home or work. Three different subjects entered rapid eye movement (REM) sleep in addition to the initial quiet sleep, as evidenced by peripheral limb twitches, erratic breathing, rapid eye movements, and self-report of dreaming. A substantial proportion of the subjects reported very deep sleep on the night of the intervention. Those subjects who did not sleep, uniformly reported a very relaxed state at the conclusion of the intervention.

### Example 8. Sleep Disordered Breathing

Two subjects, one 38 year old female, the other a 72 year old male, had obstructive sleep apnea, the male with very severe obstructive apnea (apnea hyponea index>30), documented by polysomnographic recordings at a sleep center, in addition to migraine pain. Both subjects slept during the stimulation, offering the potential to evaluate the effectiveness of the method in preventing apnea. Both subjects entered REM sleep after quiet sleep, and one subject showed repeated episodes of sleeping on multiple stimulation trials. No evidence of airway obstruction appeared in either subject. Stimulation parameters were 120 Hz vibration, 1.5 volt low level for 10 minutes, and 20 30 minutes of 3.0 volt high level. The absence of obstructive apnea, which invariable appears quickly when an individual enters quiet or REM sleep, suggests a protective role for the stimulation, as proposed by the theoretical outline above.

### Example 9. Migraine-Associated Vestibular and Visual Disturbances

A 75 year old female with migraine pain for greater than 5 years also showed severe vestibular and visual symptoms, expressed as an inability to visually focus, and an inability to maintain stability while head-turning or single-foot standing. She underwent 10 interventions using the standard parameters of 120 Hz vibration at 1.5 volts and 3.0 volts for 10 and 20 minutes, respectively. Although she was unable to show improvements in the initial intervention, pain was reduced. After 10 interventions, vestibular and visual signs improved; she was able to easily stand on one foot for >15 sec, head turning did not precipitate collapse, and visual focusing significantly improved.

### Example 10

The objective is to provide breathing and cardiovascular support in conditions which are manifested with disturbed breathing, disrupted cardiovascular control, or conditions in which both aspects of physiology are dysregulated. A second objective is to regulate autonomic outflow in a range of conditions in which disruption of autonomic regulation is a principal characteristic. During the course of studying the influence of vibratory stimulation on migraine and trigeminal pain, concurrent influences on breathing and blood pressure were found. Those influences would be useful to conditions of disturbed breathing and cardiovascular control, and are presented here.

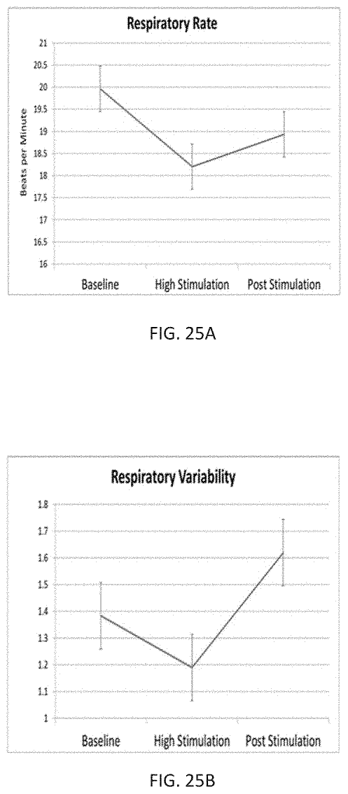

The first study (FIG. 25A, FIG. 25B) showed control of respiratory rate by continuous vibratory stimulation; respiratory rate slows, and variability decreases in 31 subjects. The study shows that cessations of breathing are reduced, i.e., fewer apnea, and that respiratory rate slows, with accompanying increased tidal volumes; oxygen saturation is maintained. Those properties could be effective in sleep-disordered breathing and heart failure conditions, among others.

FIG. 25A shows a decline in respiratory rate with auditory canal vibratory stimulation in 31 subjects over a 30-minute period. FIG. 25B shows respiratory variability declined following a no-stim baseline and during stimulation (High Stim); variability recovered post stim (Feulner et al., 2017, Am J Respir Crit Care Med; 195:A2570).

Restoration of regular breathing patterns in sleep-disturbed breathing:

A major breathing pathology in several clinical conditions is obstructive sleep apnea and periodic breathing. Periodic breathing is a respiratory pattern consisting of a burst of breathing efforts followed by a pause in both upper airway and diaphragmatic actions. The pattern is also common in patients who also show obstructive sleep apnea, with the obstructed events mixed with periodic breathing, in premature infants, where periodic breathing is also mixed with apnea of infancy, and in congenital central hypoventilation syndrome, where the pattern is often mixed with episodes of prolonged central apnea. In some conditions, such as heart failure, the pattern is exaggerated, and called “Cheyne-Stokes” breathing. Periodic breathing normally cannot be treated with continuous positive airway pressure (CPAP) devices; although CPAP is useful for obstructive sleep apnea, servo-controlled CPAP pressures are exceptionally dangerous in patients with severe periodic breathing, such as those with heart failure. The need for effective intervention is urgent; periodic breathing is exceptionally dangerous to neural tissue because the breathing pattern is a form of intermittent hypoxia, i.e., episodes of reduced oxygen followed by a return of full oxygenation. That intermittent hypoxia pattern is more destructive to neural tissue than steady-state hypoxia, since the brain appears to adapt easier to continuous lower levels of oxygen, e.g., living at altitude.

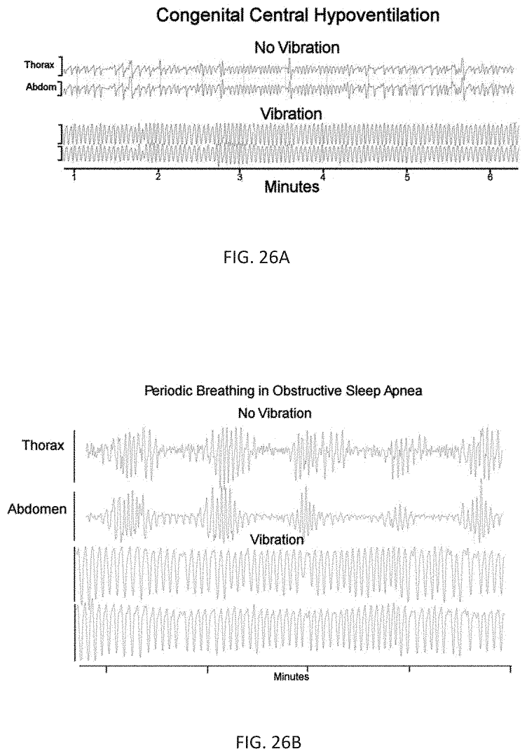

The device considered here is exceptionally successful in abolishing periodic breathing in both congenital central hypoventilation and obstructive sleep apnea, as shown in FIG. 26A and FIG. 26B.

With reference to FIG. 26A, respiratory traces of disturbed breathing in a congenital central hypoventilation (CCHS) patient in the transition to quiet sleep without vibratory stimulation showing short periods of apnea, and periodic breathing intermixed with breathing efforts. (Breathing ceases in CCHS when entering sleep; thus, ethically, only transition periods can be recorded without support). With vibratory stimulation during sleep, very regular breathing efforts occur, and no apneic events are apparent. (Patient normally uses a diaphragmatic pacer, which was turned off for this study).

With reference to FIG. 26B, obstructive sleep apnea is often accompanied by periodic breathing, a condition imposing major injury to brain structures. Abolition of periodic breathing is corrected by vibratory stimulation, avoiding the intermittent hypoxia incurred during the stopped- or minimally-breathing periods. Thoracic and abdominal movement traces; Y axis is in arbitrary units.

Correction of Hypertension; Maintenance of Blood Pressure in Sleep-Disturbed Breathing:

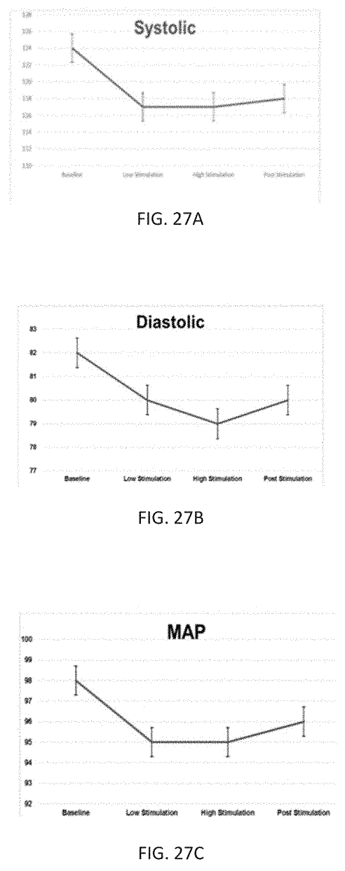

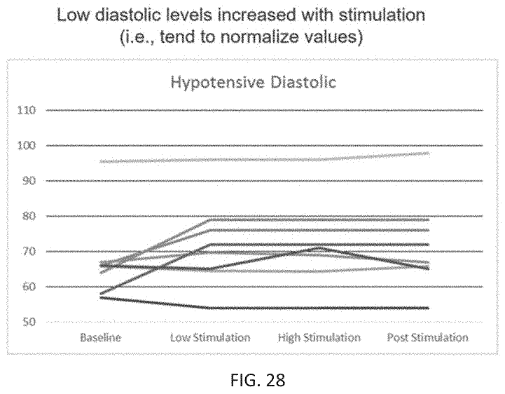

High blood pressure is reduced with vibratory stimulation to the auditory canal. FIG. 27A through FIG. 27C show the decline of systolic, diastolic, and mean blood pressure in patients with relatively high blood pressure during vibratory stimulation, while FIG. 28 shows how blood pressure is normalized in those with relatively low pressure, a significant aspect, since in some conditions, such as postural orthostatic tachycardia syndrome, few interventions exist to correct low blood pressure. The plots displaying higher blood pressure show mean and variance of 22 subjects during baseline, at low and high levels of stimulation, and after the baseline at the end of the session.

FIG. 27A through FIG. 27C show systolic, diastolic and Mean Arterial Pressure (MAP) during baseline, following low and high level vibration of the auditory canal, and after a second baseline vibration; total session time was 30 minutes.

If initial blood pressure values were initially low in patients, the vibration outcome was to normalize those values, as seen in FIG. 28. Blood pressure values showing how vibratory stimulation normalizes blood pressure in those who have mildly low blood pressure. MAP=Mean arterial pressure. The need for intervention in patients with hypotension is great; such patients frequently show syncope on sudden standing or movement, and few interventions are available.

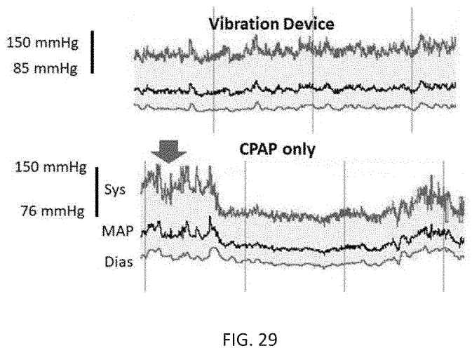

The combined data suggest that momentary blood pressure declines which accompany apnea could also be corrected. One example of that possibility is shown with a CCHS patient, who was unable to maintain blood pressure despite either positive pressure ventilation or phrenic nerve stimulation to the diaphragm. Use of the device alone (i.e., no positive ventilation or phrenic nerve stimulation) was able to support breathing near 100% oxygen saturation (CPAP was able to maintain only 90-92%, and was unable to support blood pressure), and blood pressure, which was supported adequately in the patient, significantly declined when respiratory support was switched from vibration to CPAP (Arrow, FIG. 29).

With reference now to FIG. 29, beat-by-beat systolic, mean, and diastolic blood pressure during vibration and during CPAP (onset at arrow) is shown. CPAP was unable to maintain blood pressure during sleep in this hypertensive CCHS patient (CCHS patients show very high sympathetic tone; thus, they have overall high blood pressures).

The data collectively indicate that stimulation of the cranial and cervical nerves which signal the brain for respiratory drive and timing and cardiovascular support is able to support breathing in sleep-disordered breathing conditions and conditions which require blood pressure support, and can do so simply, non-invasively, and inexpensively.

### Advantages for the Field:

The current intervention of choice for sleep-disordered breathing and for heart failure is continuous positive airway pressure (CPAP), a poorly-tolerated means with significant limitations in patient comfort and oxygen delivery; only a third of patients prescribed CPAP devices comply with sustained use. Moreover, there are significant aerosol concerns with humidification of CPAP devices and the potential for coronavirus infection. However, the most concerning issue with the CPAP device is its failure to adequately control blood pressure; patients who use CPAP over the long term have only modest management of hypertension, a critical concern in obstructive sleep apnea (Pengo M F et al., 2020, Eur Respir J., 1901945). Embodiments of the device here, however, directly manage that aspect; through stimulation of the 9^{th} nerve, which receives projections from the baroreceptors in the carotid sinus, blood pressure is maintained. The difference in outcomes can be profound; an inability to maintain blood pressure during apnea leads to loss of perfusion, with resulting hypoxemia, resulting in damage to nerve cells, fibers, and supportive glia. An unfortunate consequence is that the neural injury preferentially occurs in blood pressure regulatory areas of the brain, namely in the insular cortex, ventral lateral medulla, and deep (autonomic) fastigial nuclei of the cerebellum (Harper, R M et al., 2014, Progress in Brain Research 209:275-293; Harper, R M et al., 2013, Respiratory Physiology and Neurobiology, 188:383-391) which further leads to long-term failure to control blood pressure.

### Study Summary

Breathing patterns were evaluated, assessed by thoracic and abdominal wall movements, and beat-by-beat blood pressure, inferred from pulse transit time, following mechanical stimulation in 37 patients with obstructive or central apnea or no breathing disturbance over a 10 min baseline, 30 min stimulation, and 10 min post baseline. Blood pressure and breathing efforts were analyzed by ANCOVA (variates, sex, and age). The intervention induced sleep in over a third of patients, and abolished obstructive and periodic breathing in all subjects, creating a slow, deep, minimally-variant breathing pattern. Blood pressure in individuals with high systolic and diastolic values diminished to normative levels, while those with initial low blood pressure values increased to normative levels. Individual subjects with previously-demonstrated dramatic loss of blood pressure during sleep with modest support of breathing while using CPAP, had blood pressure restored and oxygen saturations return to near 100%. Both blood pressure and breathing can be supported in sleep-disordered breathing subjects, correcting a blood pressure concern with conventional devices.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations

What is claimed is:

1. A method for non-invasive neuromodulation comprising:
positioning a vibratory earpiece configured to contact an external ear of a subject;
applying vibrational energy through the vibratory earpiece to stimulate mechanoreceptors of sensory fibers on cranial nerves 5, 7, 9 and 10 and cervical nerves C2 and C3; and
regulating the subject's breathing and blood pressure simultaneously based on the stimulation.

2. The method of claim 1 further comprising:
regulating autonomic outflow based on the stimulation of cranial nerves which also contain parasympathetic autonomic fibers and influence sympathetic outflow.

3. The method of claim 1, wherein mechanoreceptors are stimulated simultaneously by the vibrational energy.

4. The method of claim 1, wherein the application of vibrational energy is applied through at least a portion of the skin of at least one of the auditory canal, auricle, and concha of the subject's ear.

5. The method of claim 1, wherein the stimulating is indicative of nerve sensations for airflow, upper airway muscle positioning, chemoreception, and blood pressure changes.

6. The method of claim 1, wherein the stimulating elicits reflexive motor actions to activate upper airway muscles, diaphragmatic muscles, ancillary thoracic musculature, and abdominal breathing musculature.

7. The method of claim 1, wherein the stimulating reduces blood pressure of the subject for regulating blood pressure.

8. The method of claim 1, wherein the stimulating increases blood pressure of the subject for regulating blood pressure.

9. The method of claim 1, wherein the stimulating enhances breathing extent of the subject, and reduces breathing variability.

10. The method of claim 1, wherein the stimulating regulates autonomic outflow of the subject.

11. The method of claim 1 for treating autonomic disorders.

12. The method of claim 1 for treating sleep-disturbed breathing of a periodic pattern, obstructed upper airway, central, or hypoventilatory nature.

13. The method of claim 1 for treating headache disorders.

14. The method of claim 1 for treating vestibular disorders.

15. A device for non-invasive neuromodulation comprising:
an earpiece comprising a housing configured to contact a subject's external ear;
a vibratory element connected to the housing, wherein the vibratory element transmits vibrational energy to an outer wall of housing; and
a controller configured to generate a stimulation signal for stimulating one or more mechanoreceptors of sensory fibers of cranial nerves 5, 7, 9 and 10 and cervical nerves C2 and C3 when the housing is positioned in or around the external ear canal for simultaneously regulating the subject's breathing and blood pressure.

16. The device of claim 15, wherein the stimulation signal is configured to regulate autonomic outflow.

17. The device of claim 15, wherein the stimulation signal is configured to stimulate mechanoreceptors simultaneously.

18. The device of claim 15, wherein the stimulation signal is configured to reduce blood pressure of the subject for regulating blood pressure.

19. The device of claim 15, wherein the stimulation signal is configured to increase blood pressure of the subject for regulating blood pressure.

20. The device of claim 15, wherein the stimulation signal is configured to enhance breathing of the subject.

21. The device of claim 15, wherein the vibratory element is embedded within the housing.

22. The device of claim 15, wherein the vibratory element is releasably connected to the housing.

23. A method for treating autonomic disorders or sleep-disturbed breathing comprising: positioning the device of claim 15 into or around at least one external ear canal of a subject; and applying the vibrational energy.

24. A method for treating sleep-disturbed breathing and autonomic disorders comprising:
applying vibrational stimulus to sensory nerves for simultaneous regulation of blood pressure, respiration, headache, and vestibular disorders.

25. A system for treating sleep-disturbed breathing and autonomic disorders comprising:
a vibratory element operationally coupled to a controller, wherein the controller is configured to send a stimulus signal to the vibratory element capable of stimulating sensory nerves for simultaneous regulation of blood pressure and respiration.

26. A method of reducing headache pain in a subject, comprising:
contacting the ear of a subject with device comprising a vibratory element;
transmitting vibrational energy via the device to the subject's ear; and
stimulating at least one nerve in the subject's ear via the vibrational energy, thereby reducing headache pain in the subject.
